(19) United States
(12) Patent Application Publication
(10) Pub. No.: US 2005/0123615 A1
(43) Pub. Date: Jun. 9, 2005

(54) Controlled release dosage forms of azithromycin

(75) Inventors: Roderick J. Ray, Bend, OR (US); Leah Appel, Bend, OR (US); Dwayne Friesen, Bend, OR (US); Marshall D. Crew, Bend, OR (US); David D. Newbold, Bend, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/003,855

(22) Filed: Dec. 3, 2004

Related U.S. Application Data
(60) Provisional application No. 60/527,317, filed on Dec. 4, 2003.

(51) Int. Cl. A61K 9/52; A61K 9/14

(52) U.S. Cl. 424/489; 264/5; 514/28

(57) ABSTRACT

A process for forming multiparticulates of azithromycin and a controlled release dosage form comprising multiparticulates of azithromycin and a pharmaceutically acceptable excipient are disclosed. The dosage form decreases the incidence and/or severity of GI side effects relative to currently available immediate release azithromycin dosage forms that deliver an equivalent dose. The dosage forms operate by effecting azithromycin release at a rate sufficiently slow to ameliorate side effects, yet sufficiently fast to achieve good bioavailability.

### BACKGROUND OF THE INVENTION

Azithromycin is the U.S.A.N. (generic name) for 9a-aza-9a-methyl-9-deoxo-9a-homoerythromycin A, a broad-spectrum antimicrobial compound derived from erythromycin A. U.S. Pat. Nos. 4,474,768 and 4,517,359 disclose that azithromycin and certain derivatives thereof possess antimicrobial properties and so are useful as antibiotics.

Azithromycin is available in several immediate release dosage forms. One immediate release dosage form is ZITHROMAX® tablets, which contain azithromycin dihydrate equivalent to 600 mg azithromycin. The tablets are film-coated and contain the inactive ingredients anhydrous dibasic calcium phosphate, pregelatinized starch, sodium croscarmellose, magnesium stearate and sodium lauryl sulfate.

Another immediate release dosage form is ZITHROMAX® for oral suspension, which is supplied in a single dose packet containing azithromycin dihydrate equivalent to 1 g azithromycin. The azithromycin is in the form of crystals having an approximate mean diameter of 100 μm and contains the inactive ingredients colloidal silicon dioxide, anhydrous tribasic sodium phosphate, artificial banana and cherry flavors and sucrose. The dosage form is prepared by emptying the contents of the single dose packet into 60 mL of water, mixing thoroughly, drinking the mixture immediately, adding an additional 60 mL of water to the container, mixing again to solubilize any residue and drinking the second mixture, so as to ensure consumption of the entire dosage.

It is widely known that oral dosing of azithromycin can result in the occurrence of adverse gastrointestinal (GI) side effects such as cramping, diarrhea, nausea and vomiting in a significant number of patients. Such GI side effects can also occur in non-human mammals, e.g., dogs. In combined clinical studies of azithromycin involving 3,995 human patients (all dose levels combined), 9.6% of patients reported GI side effects; the most frequent of these side effects were diarrhea (3.6%), nausea (2.6%), and abdominal pain (2.5%) Hopkins, 91 Am. J. Med. 40S (suppl 3A 1991).

The incidence of GI side effects is higher at higher doses than at lower doses. In the treatment of adult humans with a single 1 g oral dose administered in an oral suspension, the reported incidence of various GI side effects was 7% diarrhea/loose stools, 5% abdominal pain, 5% nausea and 2% vomiting (U.S. package insert for ZITHROMAX® azithromycin for oral suspension). But for a 2 g single dose administered in the same way, the reported incidence of GI side effects was 14% diarrhea/loose stools, 7% abdominal pain and 7% vomiting (Ibid). It is also known that azithromycin can cause GI side effects in non-human mammals, e.g., dogs.

Multiparticulates are a known improved dosage form of azithromycin that permit higher oral dosing with relatively reduced side effects. See commonly owned U.S. Pat. No. 6,068,859. A number of methods of formulating such azithromycin multiparticulates are disclosed in the '859 patent, including extrusion/spheronization, wax granulation, spray-drying, and spray-coating.

There is a continuing need in the art for a process of making multiparticulates containing a high degree of crystalline azithromycin. This need and others that will become apparent to one of ordinary skill in the art are met by the present invention, which is summarized and described in detail below.

### BRIEF SUMMARY OF THE INVENTION

A process for forming multiparticulates comprises the steps (a) forming a molten mixture comprising azithromycin, a pharmaceutically acceptable carrier, and an optional dissolution enhancer; (b) delivering the molten mixture of step (a) to an atomizing means to form droplets from the mixture; (c) congealing the droplets of step (b) to form the multiparticulates; and (d) post-treating the multiparticulates so as to increase the degree of crystallinity of the azithromycin in the multiparticulates. In one embodiment, the carrier has a melting point of Tₘ and step (d) comprises heating the multiparticulates to a temperature of at least about 35° C. and less than about (Tₘ° C.−10° C.). In another embodiment, the multiparticulates are formed comprising a mobility-enhancing agent. In another embodiment, the mobility-enhancing agent is added during step (a) to the molten mixture. In yet another embodiment, the mobility-enhancing agent is water. In still another embodiment, the multiparticulates are formed comprising a mobility-enhancing agent and step (d) comprises the steps: (i) placing the multiparticulates into a sealed container; and (ii) heating the sealed container to a temperature of not more than about (Tₘ° C.−10° C.).

In another aspect, the invention provides a dosage form that decreases, relative to currently available immediate release azithromycin dosage forms that deliver an equivalent dose, the incidence and/or severity of GI side effects. The dosage form can operate by effecting azithromycin release at a rate sufficiently slow to ameliorate side effects, yet sufficiently fast to achieve good bioavailability. Specific embodiments can be in (i) a sustained release oral dosage form or in (ii) a delayed release oral dosage form or in (iii) an oral dosage form which exhibits a combination of sustained and delayed release characteristics.

The multiparticulates combine good toleration with good bioavailability. In order to achieve good bioavailability when dosing azithromycin multiparticulates, the inventors believe it is necessary to control the release of the azithromycin so that the majority of the azithromycin is released within about 6 hours following ingestion. Good bioavailability is achieved because the majority of the azithromycin may be absorbed throughout the intestines. However, to reduce GI side effects, exposure of the stomach and duodenum to azithromycin is limited.

### DETAILED DESCRIPTION OF PREFERRED EMBODIMENTS

As used in the present invention, the term “about” means the specified value ±10% of the specified value.

The term “multiparticulates” refers to a multiplicity of particles, each containing azithromycin, whose totality represents the intended therapeutically useful dose of azithromycin. The term is intended to refer broadly to small particles regardless of their composition or the manner in which they are formed. The particles are small enough so that the particles travel with GI fluids to disperse in the GI fluid shortly after ingestion.

The invention is particularly useful for administering relatively large amounts of azithromycin to a patient in a single-dose or multi-dose therapy wherein the amount contained in the dosage form is preferably from about 1.5 to about 4 gA, more preferably from about 1.5 to about 3 gA, and most preferably 1.8 to 2.2 gA (“gA” means grams of active azithromycin in the dosage form). As used herein, “single dose therapy” means administering only one dose of azithromycin in the full course of therapy and “multiple dose therapy” means administering more than one dose in a single day or one or more doses over a course of two to five days or more. A daily dose can be administered from one to four times daily in equal doses. Single dose therapy is preferred. For small patients, e.g., children weighing about 30 kg or less, the multiparticulate dosage form can be scaled according to the weight of the patient; in one aspect, the dosage form contains about 30 to about 90 mgA/kg of patient body weight, preferably about 45 to about 75 mgA/kg, more preferably, about 60 mgA/kg. For veterinary applications, the dose may be adjusted to be outside these limits, depending on the size of the animal.

### Azithromycin

The multiparticulates of the present invention comprise azithromycin. Preferably, the azithromycin makes up from about 5 wt % to about 90 wt % of the total weight of the multiparticulate, more preferably from about 10 wt % to about 80 wt %, and even more preferably from about 30 wt % to about 60 wt % of the total weight of the multiparticulates.

As used herein, “azithromycin” means all amorphous and crystalline forms of azithromycin including all polymorphs, isomorphs, pseudomorphs, clathrates, salts, solvates and hydrates of azithromycin, as well as anhydrous azithromycin. Reference to azithromycin in terms of therapeutic amounts or in release rates in the claims is to active azithromycin, i.e., the non-salt, non-hydrated azalide molecule having a molecular weight of 749 g/mole.

Preferably, the azithromycin of the present invention is azithromycin dihydrate, which is disclosed in U.S. Pat. No. 6,268,489.

In alternate embodiments of the present invention, the azithromycin comprises a non-dihydrate azithromycin, a mixture of non-dihydrate azithromycins, or a mixture of azithromycin dihydrate and non-dihydrate azithromycins. Examples of suitable non-dihydrate azithromycins include, but are not limited to, alternate crystalline forms B, D, E, F, G, H, J, M, N, O, P, Q and R.

Azithromycin also occurs as Family I and Family II isomorphs, which are hydrates and/or solvates of azithromycin. The solvent molecules in the cavities have a tendency to exchange between solvent and water under specific conditions. Therefore, the solvent/water content of the isomorphs may vary to a certain extent.

Azithromycin form B, a hygroscopic hydrate of azithromycin, is disclosed in U.S. Pat. No. 4,474,768.

Azithromycin forms D, E, F, G, H, J, M, N, O, P, Q and R are disclosed in commonly owned U.S. Patent Publication No. 20030162730, published Aug. 28, 2003.

Forms B, F, G, H, J, M, N, O, and P belong to Family I azithromycin and have a monoclinic P2₁ space group with cell dimensions of a=16.3±0.3 Å, b=16.2±0.3 Å, c=18.4±0.3 Å and beta=109±2°.

Form F azithromycin is an azithromycin ethanol solvate of the formula C₃₈H₇₂N₂O₁₂.H₂O.0.5C₂H₅OH in the single crystal structure and is an azithromycin monohydrate hemi-ethanol solvate. Form F is further characterized as containing 2-5 wt % water and 1-4 wt % ethanol by weight in powder samples. The single crystal of form F is crystallized in a monoclinic space group, P2₁, with the asymmetric unit containing two azithromycin molecules, two water molecules, and one ethanol molecule, as a monohydrate/hemi-ethanolate. It is isomorphic to all Family I azithromycin crystalline forms. The theoretical water and ethanol contents are 2.3 and 2.9 wt %, respectively.

Form G azithromycin has the formula C₃₈H₇₂N₂O₁₂.1.5H₂O in the single crystal structure and is an azithromycin sesquihydrate. Form G is further characterized as containing 2.5-6 wt % water and <1 wt % organic solvent(s) by weight in powder samples. The single crystal structure of form G consists of two azithromycin molecules and three water molecules per asymmetric unit, corresponding to a sesquihydrate with a theoretical water content of 3.5 wt %. The water content of powder samples of form G ranges from about 2.5 to about 6 wt %. The total residual organic solvent is less than 1 wt % of the corresponding solvent used for crystallization.

Form H azithromycin has the formula C₃₈H₇₂N₂O₁₂.H₂O.0.5C₃H₈O₂ and may be characterized as an azithromycin monohydrate hemi-1,2 propanediol solvate.

Form H is a monohydrate/hemi-propylene glycol solvate of azithromycin free base.

Form J azithromycin has the formula C₃₈H₇₂N₂O₁₂.H₂O.0.5C₃H₇OH in the single crystal structure, and is an azithromycin monohydrate hemi-n-propanol solvate. Form J is further characterized as containing 2-5 wt % water and 1-5 wt % n-propanol by weight in powder samples. The calculated solvent content is about 3.8 wt % n-propanol and about 2.3 wt % water.

Form M azithromycin has the formula C₃₈H₇₂N₂O₁₂.H₂O.0.5C₃H₇OH, and is an azithromycin monohydrate hemi-isopropanol solvate. Form M is further characterized as containing 2-5 wt % water and 1-4 wt % 2-propanol by weight in powder samples. The single crystal structure of form M would be a monohydrate/hemi-isopropranolate.

Form N azithromycin is a mixture of isomorphs of Family I. The mixture may contain variable percentages of isomorphs F, G, H, J, M and others, and variable amounts of water and organic solvents, such as ethanol, isopropanol, n-propanol, propylene glycol, acetone, acetonitrile, butanol, pentanol, etc. The weight percent of water can range from 1-5.3 wt % and the total weight percent of organic solvents can be 2-5 wt % with each solvent making up 0.5-4 wt %.

Form O azithromycin has the formula C₃₈H₇₂N₂O₁₂.0.5H₂O.0.5C₄H₉OH, and is a hemihydrate hemi-n-butanol solvate of azithromycin free base by single crystal structural data.

Form P azithromycin has the formula C₃₈H₇₂N₂O₁₂.H₂O.0.5C₅H₁₂O and is an azithromycin monohydrate hemi-n-pentanol solvate.

Form Q is distinct from Families I and II, has the formula C₃₈H₇₂N₂O₁₂.H₂O.0.5C₄H₈O and is an azithromycin monohydrate hemi-tetrahydrofuran (THF) solvate. It contains about 4% water and about 4.5 wt % THF.

Forms D, E and R belong to Family II azithromycin and contain an orthorhombic P2₁ 2₁2₁ space group with cell dimensions of a=8.9±0.4 Å, b=12.3±0.5 Å and c=45.8±0.5 Å.

Form D azithromycin has the formula C₃₈H₇₂N₂O₁₂.H₂O.C₆H₁₂ in its single crystal structure, and is an azithromycin monohydrate monocyclohexane solvate. Form D is further characterized as containing 2-6 wt % water and 3-12 wt % cyclohexane by weight in powder samples. From single crystal data, the calculated water and cyclohexane content of form D is 2.1 and 9.9 wt %, respectively.

Form E azithromycin has the formula C₃₈H₇₂N₂O₁₂.H₂O.C₄H₈O and is an azithromycin monohydrate mono-THF solvate by single crystal analysis.

Form R azithromycin has the formula C₃₈H₇₂N₂O₁₂.H₂O.C₅H₁₂O and is an azithromycin monohydrate mono-methyl tert-butyl ether solvate. Form R has a theoretical water content of 2.1 wt % and a theoretical methyl tert-butyl ether content of 10.3 wt %.

Other examples of non-dihydrate azithromycin include, but are not limited to, an ethanol solvate of azithromycin or an isopropanol solvate of azithromycin. Examples of such ethanol and isopropanol solvates of azithromycin are disclosed in U.S. Pat. Nos. 6,365,574 and 6,245,903 and U.S. Patent Application Publication No. 20030162730, published Aug. 28, 2003.

Additional examples of non-dihydrate azithromycin include, but are not limited to, azithromycin monohydrate as disclosed in U.S. Patent Application Publication Nos. 20010047089, published Nov. 29, 2001, and 20020111318, published Aug. 15, 2002, as well as International Application Publication Nos. WO 01/00640, WO 01/49697, WO 02/10181 and WO 02/42315.

Further examples of non-dihydrate azithromycin include, but are not limited to, anhydrous azithromycin as disclosed in U.S. Patent Application Publication No. 20030139583, published Jul. 24, 2003, and U.S. Pat. No. 6,528,492.

Examples of suitable azithromycin salts include, but are not limited to, the azithromycin salts as disclosed in U.S. Pat. No. 4,474,768.

Preferably, at least 70 wt % of the azithromycin in the multiparticulates is crystalline. The degree of azithromycin crystallinity in the multiparticulates can be “substantially crystalline,” meaning that the amount of crystalline azithromycin in the multiparticulates is at least about 80%, “almost completely crystalline,” meaning that the amount of crystalline azithromycin is at least about 90%, or “essentially crystalline,” meaning that the amount of crystalline azithromycin in the multiparticulates is at least 95%.

The crystallinity of azithromycin in the multiparticulates may be determined using Powder X Ray Diffraction (PXRD) analysis. In an exemplary procedure, PXRD analysis may be performed on a Bruker AXS D8 Advance diffractometer. In this analysis, samples of about 500 mg are packed in Lucite sample cups and the sample surface smoothed using a glass microscope slide to provide a consistently smooth sample surface that is level with the top of the sample cup. Samples are spun in the (p plane at a rate of 30 rpm to minimize crystal orientation effects. The X-ray source (S/B KCu_{α}, λ=1.54 Å) is operated at a voltage of 45 kV and a current of 40 mA. Data for each sample are collected over a period of from about 20 to about 60 minutes in continuous detector scan mode at a scan speed of about 12 seconds/step and a step size of 0.02°/step. Diffractograms are collected over the 2θ range of 10° to 16°.

The crystallinity of the test sample is determined by comparison with calibration standards as follows. The calibration standards consist of physical mixtures of 20 wt %/80 wt % azithromycin/carrier, and 80 wt %/20 wt % azithromycin/carrier. Each physical mixture is blended together 15 minutes on a Turbula mixer. Using the instrument software, the area under the diffractogram curve is integrated over the 20 range of 10° to 16° using a linear baseline. This integration range includes as many azithromycin-specific peaks as possible while excluding carrier-related peaks. In addition, the large azithromycin-specific peak at approximately 10° 2θ is omitted due to the large scan-to-scan variability in its integrated area. A linear calibration curve of percent crystalline azithromycin versus the area under the diffractogram curve is generated from the calibration standards. The crystallinity of the test sample is then determined using these calibration results and the area under the curve for the test sample. Results are reported as a mean percent azithromycin crystallinity (by crystal mass).

### Release Rates

Multiparticulate dosage forms that are within the scope of the invention provide a controlled azithromycin release rate that is fast enough to achieve good bioavailability, but slow enough to limit the drug's exposure to the stomach and duodenum. To obtain good toleration, the dosage form should limit the amount of azithromycin released to the stomach. In addition, the dosage form should release azithromycin in a controlled manner upon discharge from the stomach so that the concentration of azithromycin in the duodenum remains low. At the same time, the rate of release of azithromycin should be sufficiently high to obtain good bioavailability.

While not wishing to be bound by any particular theory or mechanism, it is believed that when a patient ingests a multiparticulate dosage form, the multiparticulates disperse throughout the stomach. Because of their small size, the multiparticulates leave the stomach at roughly the same rate as liquids. As a result, there is a broad distribution of residence times for the multiparticulates in the stomach, with some residing in the stomach for only a few minutes, and others remaining in the stomach for as long as 60 minutes or more, depending on the rate at which liquid enters and leaves the stomach. Thus, at any given point in time shortly after ingestion of the dosage form and for the next few hours, a portion of the multiparticulates will be in the stomach, a portion in the duodenum, and a portion in the small intestine beyond the duodenum. Thus, unlike non-multiparticulate dosage forms, where essentially all of the dosage form will travel as a unit from the stomach to the duodenum and then to the small intestine, multiparticulates are distributed over a larger portion of the GI tract shortly after ingestion.

Because of this distribution, the maximum concentration of azithromycin at any point in the GI tract will be lower when a multiparticulate dosage form is ingested than when a non-multiparticulate dosage form having the same azithromycin release rate is ingested. It is believed that the lower maximum concentration of azithromycin in the GI tract provided by the multiparticulates leads to improved toleration. Alternatively, a faster azithromycin release rate from a multiparticulate dosage form may provide the same toleration as a non-multiparticulate dosage form with a slower rate of release. This same fast rate of release would result in poor toleration for a non-multiparticulate dosage form.

In order to verify improvement in toleration it is helpful to identify and quantify negative side effects that can accompany azithromycin therapy. Thus, “percentage adverse events” is defined as the percentage of subjects in an in vivo test that experience GI adverse events related to the ingestion of an azithromycin dosage form. “GI adverse events” is defined as nausea, diarrhea, abdominal pain and vomiting. By way of example, in an in vivo test, if 10 subjects out of 20 experience GI adverse events, the percentage adverse events would be 100×(10÷20), or 50%. Percentage adverse events may be based on one or more or on all GI adverse events observed; thus, in an in vivo test, if 7 subjects out of 20 experience nausea, the percentage adverse events would be 100×(7÷20), or 35%.

A “relative degree of improvement in toleration” is defined as the ratio of (1) the percentage adverse events arising from the administration of an immediate release control dosage form to (2) the percentage adverse events arising from the administration of a controlled release multiparticulate dosage form of the present invention, where the immediate release control dosage form and the controlled release multiparticulate dosage form contain the same amount of azithromycin. The immediate release control dosage form may be any conventional immediate release dosage form, such as ZITHROMAX® tablets, or single-dose packets for oral suspension. For example, if an immediate release control dosage form provides a percentage adverse events arising from the administration of 20% while the multiparticulate dosage form of the present invention provides a percentage adverse events arising from the administration of 10%, then the relative degree of improvement in toleration is 20%+10% or 2. In one aspect, the multiparticulate dosage forms of the present invention provide a relative degree of improvement in toleration of at least 1.1 relative to an immediate release control dosage form, preferably of at least 1.25, more preferably at least 1.5, even more preferably at least 2.0, and most preferably at least 3.0. Relative degrees of improvement in toleration of 5 or more can be obtained with the azithromycin multiparticulate dosage forms of the present invention.

### Rate of Release in the Stomach

In a separate aspect of the invention, the multiparticulate dosage forms control the rate of release of azithromycin in the stomach. High relative degrees of improvement in toleration can be obtained by limiting azithromycin exposure to the stomach.

The solubility of azithromycin in water is highly pH-dependent, with the solubility at low pH (such as at the pH of the stomach) being much higher than at high pH (such as at the pH of the duodenum and lower portion of the small intestine). Because of this, the azithromycin release rate from a multiparticulate dosage form will tend to be higher at low pH than at high pH. Thus, one method to keep the amount of azithromycin released in the stomach at an acceptable level is to include an effective amount of an alkalizing agent, such as a base or buffer, in the multiparticulate dosage form so as to temporarily increase the pH of the stomach. Alkalizing agents include, for example, antacids as well as other pharmaceutically acceptable (1) organic and inorganic bases, (2) salts of strong organic and inorganic acids, (3) salts of weak organic and inorganic acids, and (4) buffers.

Examples of such alkalizing agents include, but are not limited to, aluminum salts such as magnesium aluminum silicate; magnesium salts such as magnesium carbonate, magnesium trisilicate, magnesium aluminum silicate, magnesium stearate; calcium salts such as calcium carbonate; bicarbonates such as calcium bicarbonate and sodium bicarbonate; phosphates such as monobasic calcium phosphate, dibasic calcium phosphate, dibasic sodium phosphate, tribasic sodium phosphate (TSP), dibasic potassium phosphate, tribasic potassium phosphate; metal hydroxides such as aluminum hydroxide, sodium hydroxide and magnesium hydroxide; metal oxides such as magnesium oxide; N-methyl glucamine; arginine and salts thereof; amines such as monoethanolamine, diethanolamine, triethanolamine, and tris(hydroxymethyl)aminomethane (TRIS); and combinations thereof.

Preferably, the alkalizing agent is TRIS, magnesium hydroxide, magnesium oxide, dibasic sodium phosphate, TSP, dibasic potassium phosphate, tribasic potassium phosphate or a combination thereof. More preferably, the alkalizing agent is a combination of TSP and magnesium hydroxide.

An “effective amount” of an alkalizing agent means an amount of one or more alkalizing agents which, when administered in combination with azithromycin, provides an improvement in the percentage of recipients tolerating azithromycin administration, without GI side effects, relative to a control dosage form containing the same amount of active azithromycin. In this connection, the minimum amount of alkalizing agent suitable to constitute an effective amount is that amount which would result in a relative degree of improvement in azithromycin toleration of at least 1.1, disclosed more fully in commonly assigned U.S. patent application Ser. No. ______ (“Azithromycin Dosage Forms With Reduced Side Effects,” Attorney Docket No. PC25240), filed concurrently herewith.

Alternatively, an effective amount of an alkalizing agent can be determined in the following in vitro test. First, a 20-mL sample of 0.1 N HCl is placed in an appropriate container. Second, the candidate alkalizing agent is added to 60 mL of water. The so-formed alkalizing agent solution is then added to the 20-mL sample of 0.1 N HCl and the pH of the resulting solution is monitored over time. When the azithromycin is in the form of sustained-release multiparticulates, an effective amount of alkalizing agent is one that causes the pH of the solution to be at least 5, preferably at least 6, and more preferably at least 7.

An in vivo test may be used to determine if a multiparticulate dosage form is within the scope of the invention with respect to the rate of drug released in the stomach. Alternatively, multiparticulates may be evaluated in an in vitro dissolution test. A suitable in vitro dissolution test is described below.

The following terms are used in the dissolution tests herein:

“Administration” refers generally to introducing the dosage form to a use environment, either by placing the dosage form in an in vitro dissolution medium or by ingestion by a patient so as to enter the in vivo environment of the GI tract.

“Use environment” can be either the in vivo environment of the GI tract of a mammal and particularly a human, or the in vitro environment of either an acidic or a buffered test medium, as outlined below.

“Use environment” can be either the in vivo environment of the GI tract of a mammal and particularly a human, or the in vitro environment of a test solution. Exemplary test solutions include aqueous solutions at 37° C. comprising (1) 0.1 N HCl, simulating gastric fluid without enzymes; (2) 0.01 N HCl, simulating gastric fluid that avoids excessive acid degradation of azithromycin, and (3) 50 mM KH₂PO₄, adjusted to pH 6.8 using KOH or 50 mM Na₃PO₄, adjusted to pH 6.8 using NaOH, both of which simulate intestinal fluid without enzymes. The inventors have also found that for some formulations, an in vitro test solution comprising 100 mM Na₂HPO₄, adjusted to pH 6.0 using NaOH provides a discriminating means to differentiate among different formulations on the basis of dissolution profile. It has been determined that in vitro dissolution tests in such solutions provide a good indicator of in vivo performance and bioavailability. Further details of in vitro tests and test solutions are described herein.

To determine whether a multiparticulate dosage form releases azithromycin sufficiently slowly in the stomach to come within the scope of the invention, the following acidic dissolution test may be performed. A sample of the dosage form containing about 2 gA of azithromycin is administered to an acidic dissolution test medium consisting of 750 mL of 0.01 N HCl at 37° C. in a USP rotating paddle apparatus of the type described in USP XXIII dissolution test chapter 711, where the paddles rotate at 50 rpm. At 15 minutes (0.25 hour) after administration of the dosage form to the test medium, an aliquot of the test medium is removed. The aliquot is filtered using a 0.45-μm syringe filter prior to analyzing via High Performance Liquid Chromatography (HPLC) (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer).

For smaller doses, such as doses for children and small adults, the volume of the acidic dissolution test medium is reduced proportionately according to the size of the dose used. For example, a pediatric dose of 1 gA is 50% of the 2000 mgA dose described above. Thus, in this case, the 750 mL volume of the buffered dissolution test medium used for the 2 gA dose is reduced by 50%, or to 375 mL. Applying this prorata concept, one skilled in the art may readily determine the appropriate amount of acidic dissolution test medium to use based on the dose of azithromycin present in the dosage form.

When the dosage form is a tablet or capsule, either may be placed directly in the test medium. If the dosage form is provided as a paste, slurry, or suspension, this may also be placed directly in the test medium. If the dosage form is a powder or granules, it may first be suspended or mixed with water or some other liquid, including a portion of the test medium. In such cases, the dosage form is usually provided with instructions for dosing, which should be followed when administering the dosage form to the test medium.

### Rate of Release in the Duodenum and Small Intestine

In another separate aspect of the invention, the multiparticulate dosage forms control the rate of release of azithromycin in the duodenum and small intestine wherein the pH is greater than in the stomach. Once the multiparticulates exit the stomach, they enter the duodenum. The inventors have determined that the rate of release of azithromycin from the multiparticulates while in the duodenum should be controlled to provide an improvement in toleration.

At the same time, the azithromycin release rate from the multiparticulates after entering the duodenum should be sufficiently fast that the multiparticulate dosage form provides a high bioavailability relative to an immediate release control composition consisting essentially of the same amount of azithromycin but in an immediate-release dosage form. Preferably, the multiparticulates provide a bioavailability of at least 60%, more preferably at least 70%, even more preferably at least 80%, and most preferably at least 90% relative to the control composition.

The control composition is preferably ZITHROMAX® for oral suspension, supplied in a single dose packet. Each packet contains azithromycin dihydrate equivalent to 1000 mgA azithromycin and the inactive ingredients previously noted, including about 88 mg of anhydrous tribasic sodium phosphate. An equivalent quantity of azithromycin should be used in both the control and test compositions. Thus, if the inventive multiparticulate dosage form contains 2000 mgA azithromycin, the control composition should also contain 2000 mgA azithromycin. Alternatively, the control can consist of ZITHROMAX® tablets.

An in vivo test, such as a crossover study, may be used to determine whether a multiparticulate dosage form is within the scope of this invention with respect to a sufficiently slow release of azithromycin in the duodenum and small intestine that still meets the bioavailability requirement. In an in vivo crossover study a “test dosage form” is dosed to half a group of test subjects and, after an appropriate washout period (e.g., two weeks) the same subjects are dosed with an “immediate release control dosage form.” The “immediate release control dosage form” is the ZITHROMAX® for oral suspension described above, containing an equivalent quantity of azithromycin. The other half of the group is dosed with the control dosage form first followed by the test dosage form. The concentration of azithromycin in the duodenum can be determined by sampling the contents of the duodenum using, for example, a nasoenteric tube. The placement of the tube in the duodenum can be confirmed by fluoroscopy. The concentration of azithromycin in the duodenum can then be determined as a function of time for each subject.

Due to the inherent difficulties in performing this test, the concentration of azithromycin in the blood (serum or plasma) may instead be used as an indicator of the concentration of drug in the duodenum. While the concentration of azithromycin in the blood will also reflect absorption of drug in the stomach and the lower portion of the small intestine, the inventors believe that the concentration of drug in the blood may be used as an indicator of the concentration of drug in the duodenum. In one embodiment, a dosage form is considered to be a part of this aspect of the invention if the ratio of (1) the maximum concentration of drug in the blood provided by the dosage form of the present invention to (2) the maximum concentration of drug in the blood provided by an immediate release control dosage form containing the same amount of azithromycin is less than 0.95. This ratio is preferably determined for each subject and then the ratio averaged over all the subjects in the study. Preferably, the ratio is less than 0.8, and more preferably less than 0.67.

Relative bioavailability is measured as the azithromycin concentration in the blood (serum or plasma) versus time area under the curve (AUC) determined for the test group divided by the AUC provided by the control dosage form. Preferably, this test/control ratio is determined for each subject, and then the ratios are averaged over all subjects in the study. In vivo determinations of AUC can be made by plotting the serum or plasma concentration of azithromycin along the ordinate (y-axis) against time along the abscissa α-axis). The determination of AUCs is a well-known procedure and is described, for example, in Welling, “Pharmacokinetics Processes and Mathematics,” ACS Monograph 185 (1986).

Due to the inherent difficulties in performing such in vivo tests, multiparticulate dosage forms are instead preferably evaluated by the following in vitro test to determine if they release azithromycin to the duodenum and small intestine sufficiently slowly yet meet the bioavailability requirement to come within the scope of the invention.

A sample adult dose of the multiparticulate dosage form, typically comprising 2 gA azithromycin, is administered to a buffered dissolution test medium consisting essentially of 900 mL of pH 6.0 Na₂HPO₄ buffer at 37° C. in a USP rotating paddle apparatus of the type described in USP XXIII dissolution test chapter 711, where the paddles rotate at 50 rpm. At 15 minutes, 30 minutes, and 1 hour following test initiation, i.e., administration of the dosage form to the buffered test medium, filtered aliquots (typically 5 or 10 mL) from the test medium are analyzed for azithromycin by HPLC as described above. Dissolution results are reported as weight percent (wt %) of total dose of active azithromycin dissolved versus time, or as mgA dissolved versus time where a particular dose is specified.

For doses of less than about 2 g, such as doses for children and small adults, the volume of the buffered dissolution test medium is reduced proportionately based on the size of the dose used. For example, a pediatric dose of 1 gA is 50% of the 2 gA dose described above. Thus, in this case, the 900 mL volume of the buffered dissolution test medium used for the 2 gA dose is reduced by 50%, or to 450 mL. Applying this prorata concept, one skilled in the art will readily determine the appropriate amount of buffered test medium to use based on the dose of azithromycin present in the dosage form.

The multiparticulate dosage forms achieve the combination of good toleration and bioavailability by controlling release of the azithromycin from the dosage form within a range of release rates.

Multiparticulate dosage forms of the present invention containing 1.5 gA to 7 gA azithromycin, following administration to a stirred buffered test medium comprising 900 mL of pH 6.0 Na₂HPO₄ buffer at 37° C., release azithromycin to the test medium at the following rate: (i) from about 15 to about 55 wt %, but no more than 1.1 gA of the azithromycin in the dosage form at 0.25 hour; (ii) from about 30 to about 75 wt %, but no more than 1.5 gA, preferably no more than 1.3 gA of the azithromycin in the dosage form at 0.5 hour; and (iii) greater than about 50 wt % of the azithromycin in the dosage form at 1 hour after administration to the buffered test medium. For doses below 1.5 gA, such as pediatric doses, the dose should be scaled up to 2 gA and then evaluated using this in vitro test.

The dosage forms do not provide immediate release of azithromycin, but rather provide controlled release of azithromycin to the use environment. By “immediate release” is meant a dosage form that releases more than about 75 wt % of the contained azithromycin within one half hour or less following administration to a use environment. By “controlled release” is meant that the release of azithromycin is slower than immediate release. As used herein, “controlled release” refers to both sustained release and delayed release. Optionally, a portion of the azithromycin in the dosage form may be released immediately, provided the overall rate of release of azithromycin from the dosage form is within the scope of the release rates described herein. The rate of release noted above is slower than that provided by a commercial immediate release dosage form, such as ZITHROMAX® tablets, or single-dose packets for oral suspension.

In a preferred embodiment of the invention, the multiparticulate dosage forms control the release of azithromycin in both the stomach and in the duodenum and small intestine. In this embodiment, the multiparticulate dosage forms meet the release rates described above for release in the stomach, and also meet the release rates described herein for release in the duodenum and small intestine. The multiparticulate dosage forms may be evaluated using either the in vivo or in vitro release rates described above.

Preferably, the dosage form provides a relative bioavailability of at least 60%, more preferably at least 70%, and even more preferably at least 80%, as well as a relative degree of improvement in toleration that is at least 1.1, preferably at least 1.25, all relative to an immediate release control dosage form containing an equivalent quantity of azithromycin. Relative bioavailability may be calculated based on blood level over the first 24 hours, or over longer periods of time.

### Dosage Forms

The multiparticulate dosage form may be supplied in one of several forms, all well known in the art. Exemplary dosage forms include: tablets; capsules; pills; powders or granules that may be taken orally either dry or reconstituted by addition of water or other liquids to form a paste, slurry, suspension or solution; and a unit dose packet, sometimes referred to as a “sachet”. Various additives may be mixed, ground, or granulated with the multiparticulates to form a suitable dosage form. The term “dosage form” includes a plurality of devices that collectively deliver the desired amount of multiparticulates, preferably administered within about 60 minutes of one another to achieve a desired dose of azithromycin. In all cases, following administration to the use environment, the dosage form releases the multiparticulates so that they are substantially dispersed therein. When the use environment is the GI tract of a patient, the multiparticulates disperse in the stomach and then leave the stomach with gastric fluids. Any dosage form that operates in this manner is considered to be within the scope of the invention.

Although the key ingredient present in the dosage form is simply the multiparticulates, the inclusion of other excipients in the composition may be useful.

As previously noted, the dosage form preferably includes an effective amount of an alkalizing agent to temporarily increase the pH of the stomach to the degree noted above, thereby reducing the rate at which azithromycin is released in the stomach. The amount of alkalizing agent to add depends on several factors, including the fed state of the patient, the dissociation constant of the alkalizing agent, and the ionic strength of the stomach contents.

One very useful class of excipients consists of surfactants. Suitable surfactants include fatty acid and alkyl sulfonates; commercial surfactants such as benzalkonium chloride (HYAMINE® 1622, available from Lonza, Inc., Fairlawn, N.J.); dioctyl sodium sulfosuccinate (DOCUSATE SODIUM™, available from Mallinckrodt Specialty Chemicals, St. Louis, Mo.); polyoxyethylene sorbitan fatty acid esters (TWEEN®, available from ICI Americas Inc., Wilmington, Del.; LIPOSORB® P-20, available from Lipochem Inc., Patterson N.J.; CAPMUL® POE-0, available from Abitec Corp., Janesville, Wis.); and natural surfactants such as sodium taurocholic acid, 1-palmitoyl-2-oleoyl-sn-glycero-3-phosphocholine, lecithin, and other phospholipids and mono- and di-alkyl glycerides. Such materials can advantageously be employed to increase the rate at which the multiparticulates disperse when administered to the use environment.

Conventional matrix materials, fillers, diluents, lubricants, preservatives, thickeners, anticaking agents, disintegrants, or binders may also be included in the dosage form.

Examples of matrix materials, fillers, or diluents include lactose, mannitol, xylitol, microcrystalline cellulose, dibasic calcium phosphate and starch.

Examples of disintegrants include sodium starch glycolate, sodium alginate, carboxymethylcellulose sodium, methyl cellulose, croscarmellose sodium and crosslinked forms of polyvinyl pyrrolidone, also known as crospovidone.

Examples of binders include methyl cellulose, microcrystalline cellulose, starch, and gums such as guar gum, and tragacanth.

Examples of lubricants include magnesium stearate, calcium stearate, and stearic acid.

Examples of preservatives include sulfites (an antioxidant), benzalkonium chloride, methyl paraben, propyl paraben, benzyl alcohol and sodium benzoate.

Examples of suspending agents or thickeners include xanthan gum, starch, guar gum, sodium alginate, carboxymethyl cellulose, sodium carboxymethyl cellulose, methyl cellulose, hydroxypropyl methyl cellulose, polyacrylic acid, silica gel, aluminum silicate, magnesium silicate, and titanium dioxide.

Examples of anticaking agents or fillers include colloidal silicon oxide and lactose.

Other conventional excipients may be employed in the compositions of this invention, including those excipients well-known in the art. Generally, excipients such as pigments, lubricants, flavorants, and so forth may be used for customary purposes and in typical amounts without adversely affecting the properties of the compositions.

In one embodiment, the dosage form is in the form of a tablet. The term “tablet” is intended to embrace compressed tablets, coated tablets, and other forms known in the art. See for example, Remington's Pharmaceutical Sciences (18th Ed. 1990). Upon administration to the use environment, the tablet rapidly disintegrates, allowing the multiparticulates to be dispersed in the use environment.

In one embodiment, the tablet comprises multiparticulates that have been mixed with a binder, disintegrants, or other excipients known in the art, and then formed into a tablet using compressive forces. Examples of binders include microcrystalline cellulose, starch, gelatin, polyvinyl pyrrolidinone, polyethylene glycol, and sugars such as sucrose, glucose, dextrose, and lactose. Examples of disintegrants include sodium starch glycolate, croscarmellose sodium, crospovidone, and sodium carboxymethyl cellulose. The tablet may also include an effervescent agent (acid-base combinations) that generates carbon dioxide when placed in the use environment. The carbon dioxide generated helps in disintegration of the tablet. Other excipients, such as those discussed above, may also be included in the tablet.

The multiparticulates, binder, and other excipients used in the tablet may be granulated prior to formation of the tablet. Wet- or dry-granulation processes, well known in the art, may be used, provided the granulation process does not change the release profile of the multiparticulates. Alternatively, the materials may be formed into a tablet by direct compression.

The compression forces used to form the tablet should be sufficiently high to provide a tablet with high strength, but not too high to damage the multiparticulates contained in the tablet. Generally, compression forces that result in tablets with a hardness of about 3 to about 10 Kp are desired.

Alternatively, tablets may also be made using non-compression processes. In one embodiment, the tablet is formed by a lyophylization process. In this process the multiparticulates are mixed with an aqueous solution or paste of water-soluble excipients and placed into a mold. The water is then removed by lyophylization, resulting in a highly porous, fast dissolving tablet containing the multiparticulates. Examples of water-soluble excipients used in such tablets include gelatin, dextran, dextrin, polyvinyl pyrrolidone, polyvinyl alcohol, trehalose, xylitol, sorbitol and mannitol.

In another embodiment, the dosage form is in the form of a capsule, well known in the art. See Remington's Pharmaceutical Sciences (18th Ed. 1990). The term “capsule” is intended to embrace solid dosage forms in which the multiparticulates and optional excipients are enclosed in either a hard or soft, soluble container or shell. Upon administration to the use environment, the shell dissolves or disintegrates, releasing the contents of the capsule to the use environment. The hard gelatin capsule, typically made from gelatin, consists of two sections, one slipping over the other. The capsules are made by first blending the multiparticulates and optional excipients, such as those listed above. The ingredients may be granulated using wet- or dry-granulation techniques to improve the flow of the fill material. The capsules are filled by introducing the fill material into the longer end or body of the capsule and then slipping on the cap. For soft-gelatin capsules, the fill material may first be suspended in an oil or liquid prior to filling the capsule.

The dosage form may also be in the form of pills. The term “pill” is intended to embrace small, round solid dosage forms that comprise the multiparticulates mixed with a binder and other excipients as described above. Upon administration to the use environment, the pill rapidly disintegrates, allowing the multiparticulates to be dispersed therein.

In another embodiment, the multiparticulate dosage form is in the form of a powder or granules comprising the multiparticulates and other excipients as described above, that is then suspended in a liquid dosing vehicle, including an aqueous dosing vehicle, prior to dosing. Such dosage forms may be prepared by several methods. In one method, the powder is placed into a container and an amount of a liquid, such as water, is added to the container. The container is then mixed, stirred, or shaken to suspend the dosage form in the water. In another method, the multiparticulates and dosing vehicle excipients are supplied in two or more separate packages. The dosing vehicle excipients are first dissolved or suspended in a liquid, such as water, and then the multiparticulates are added to the liquid vehicle solution. Alternatively, the dosing vehicle excipients and multiparticulates, in two or more individual packages, can be added to the container first, water added to the container, and the container mixed or stirred to form a suspension.

Water is an example of a liquid that can be used to form the dosage form of the invention. Other liquids may also be used and are intended to be within the scope of the invention. Examples of suitable liquids include beverages, such as coffee, tea, milk, and various juices. Also included is water mixed with other excipients to help form the dosage form, including surfactants, thickeners, suspending agents, and the like.

The multiparticulate dosage form may also be in the form of a dosing straw or other such device that allows the patient to sip water or other liquid through the device, the device being designed to mix the liquid with the powdered or granular dosage form contained in the device.

The multiparticulate dosage form may also be in the form of a paste, slurry or suspension.

In one embodiment, the multiparticulate dosage form comprises controlled release multiparticulates, a sweetener, an alkalizing agent, an anticaking agent, a viscosity-enhancing agent and a flavorant.

### Processes for Making Multiparticulates

The multiparticulates can be made by any known process that results in particles with the desired size and release rate characteristics for the azithromycin. In one embodiment, the multiparticulate comprises azithromycin and a pharmaceutically acceptable carrier. By “pharmaceutically acceptable” is meant the carrier must be compatible with the other ingredients of the composition, and not deleterious to the recipient thereof. The carrier functions as a matrix for the multiparticulate and to affect the rate of release of azithromycin from the multiparticulate. The carrier may be a single material or a mixture of two or more materials.

Preferred processes for forming such multiparticulates include thermal-based processes, such as melt- and spray-congealing; liquid-based processes, such as extrusion spheronization, wet granulation, spray-coating, and spray-drying; and other granulation processes such as dry granulation and melt granulation.

The multiparticulates generally have a mean diameter of less than about 5000 μm, preferably less than 3000 μm, and most preferably less than about 1000 μm.

In a preferred embodiment, the mean diameter of the multiparticulates ranges from about 40 to about 3000 μm, preferably from about 50 to about 1000 μm, and most preferably from about 100 to about 300 μm. Note that the diameter of the multiparticulates can be used to adjust the release rate of azithromycin from the multiparticulates. Generally, the smaller the diameter of the multiparticulates, the faster will be the azithromycin release rate from a particular multiparticulate formulation. This is because the overall surface area in contact with the dissolution medium increases as the diameter of the multiparticulates decreases. Thus, adjustments in the mean diameter of the multiparticulates can be used to adjust the azithromycin release profile.

The multiparticulates may be made by a melt-congeal process comprising the steps of (a) forming a molten mixture comprising azithromycin and a pharmaceutically acceptable carrier; (b) delivering the molten mixture of step (a) to an atomizing means to form droplets from the molten mixture; and (c) congealing the droplets from step (b) to form the multiparticulates.

The azithromycin in the molten mixture may be dissolved in the molten mixture, may be a suspension of crystalline azithromycin distributed in the molten mixture, or any combination of such states or those states that are in between. Preferably, the molten mixture comprises a homogeneous suspension of crystalline azithromycin in the molten carrier where the fraction of azithromycin that melts or dissolves in the molten carrier is kept relatively low. Preferably less than about 30 wt % of the total azithromycin melts or dissolves in the molten carrier. It is preferred that the azithromycin be present as the crystalline dihydrate.

Thus, by “molten mixture” is meant that the mixture of azithromycin and carrier are heated sufficiently that the mixture becomes sufficiently fluid that the mixture may be formed into droplets or atomized. Atomization of the molten mixture may be carried out using any of the atomization methods described below. Generally, the mixture is molten in the sense that it will flow when subjected to one or more forces such as pressure, shear, and centrifugal force, such as that exerted by a centrifugal or spinning-disk atomizer. Thus, the azithromycin/carrier mixture may be considered “molten” when the mixture, as a whole, is sufficiently fluid that it may be atomized. Generally, a mixture is sufficiently fluid for atomization when the viscosity of the molten mixture is less than about 20,000 cp, preferably less than about 15,000 cp, more preferably less than about 10,000 cp. Often, the mixture becomes molten when the mixture is heated above the melting point of one or more of the carrier components, in cases where the carrier is sufficiently crystalline to have a relatively sharp melting point; or, when the carrier components are amorphous, above the softening point of one or more of the carrier components. Thus, the molten mixture is often a suspension of solid particles in a fluid matrix. In one preferred embodiment, the molten mixture comprises a mixture of substantially crystalline azithromycin particles suspended in a carrier that is substantially fluid. In such cases, a portion of the azithromycin may be dissolved in the fluid carrier and a portion of the carrier may remain solid.

Although the term “melt” refers specifically to the transition of a crystalline material from its crystalline to its liquid state, which occurs at its melting point, and the term “molten” refers to such a crystalline material in its liquid state, as used herein, the terms are used more broadly, referring in the case of “melt” to the heating of any material or mixture of materials sufficiently that it becomes fluid in the sense that it may be pumped or atomized in a manner similar to a crystalline material in the liquid state. Likewise “molten” refers to any material or mixture of materials that is in such a fluid state.

Virtually any process can be used to form the molten mixture. One method involves melting the carrier in a tank, adding the azithromycin to the molten carrier, and then mixing the mixture to ensure the azithromycin is uniformly distributed therein. Alternatively, both the azithromycin and carrier may be added to the tank and the mixture heated and mixed to form the molten mixture. When the carrier comprises more than one material, the molten mixture may be prepared using two tanks, melting a first carrier in one tank and a second in another. The azithromycin is added to one of these tanks and mixed as described above. In another method, a continuously stirred tank system may be used, wherein the azithromycin and carrier are continuously added to a heated tank equipped with means for continuous mixing, while the molten mixture is continuously removed from the tank.

The molten mixture may also be formed using a continuous mill, such as a Dyno® Mill. The azithromycin and carrier are typically fed to the continuous mill in solid form, entering a grinding chamber containing grinding media, such as beads 0.25 to 5 mm in diameter. The grinding chamber typically is jacketed so heating or cooling fluid may be circulated around the chamber to control its temperature. The molten mixture is formed in the grinding chamber, and exits the chamber through a separator to remove the grinding media.

An especially preferred method of forming the molten mixture is by an extruder. By “extruder” is meant a device or collection of devices that creates a molten extrudate by heat and/or shear forces and/or produces a uniformly mixed extrudate from a solid and/or liquid (e.g., molten) feed. Such devices include, but are not limited to single-screw extruders; twin-screw extruders, including co-rotating, counter-rotating, intermeshing, and non-intermeshing extruders; multiple screw extruders; ram extruders, consisting of a heated cylinder and a piston for extruding the molten feed; gear-pump extruders, consisting of a heated gear pump, generally counter-rotating, that simultaneously heats and pumps the molten feed; and conveyer extruders. Conveyer extruders comprise a conveyer means for transporting solid and/or powdered feeds, such as a screw conveyer or pneumatic conveyer, and a pump. At least a portion of the conveyer means is heated to a sufficiently high temperature to produce the molten mixture. The molten mixture may optionally be directed to an accumulation tank, before being directed to a pump, which directs the molten mixture to an atomizer. Optionally, an in-line mixer may be used before or after the pump to ensure the molten mixture is substantially homogeneous. In each of these extruders the molten mixture is mixed to form a uniformly mixed extrudate. Such mixing may be accomplished by various mechanical and processing means, including mixing elements, kneading elements, and shear mixing by backflow. Thus, in such devices, the composition is fed to the extruder, which produces a molten mixture that can be directed to the atomizer.

Once the molten mixture has been formed, it is delivered to an atomizer that breaks the molten mixture into small droplets. Virtually any method can be used to deliver the molten mixture to the atomizer, including the use of pumps and various types of pneumatic devices such as pressurized vessels or piston pots. When an extruder is used to form the molten mixture, the extruder itself can be used to deliver the molten mixture to the atomizer. Typically, the molten mixture is maintained at an elevated temperature while delivering the mixture to the atomizer to prevent solidification of the mixture and to keep the molten mixture flowing.

Generally, atomization occurs in one of several ways, including (1) by “pressure” or single-fluid nozzles; (2) by two-fluid nozzles; (3) by centrifugal or spinning-disk atomizers; (4) by ultrasonic nozzles; and (5) by mechanical vibrating nozzles. Detailed descriptions of atomization processes can be found in Lefebvre, Atomization and Sprays (1989) or in Perry's Chemical Engineers' Handbook (7th Ed. 1997). In a preferred embodiment, the atomizer is a centrifugal or spinning-disk atomizer, such as the FX1 100-mm rotary atomizer manufactured by Niro A/S of Soeborg, Denmark.

Once the molten mixture has been atomized, the droplets are congealed, typically by contact with a gas or liquid at a temperature below the solidification temperature of the droplets. Typically, it is desirable that the droplets are congealed in less than about 60 seconds, preferably in less than about 10 seconds, more preferably in less than about 1 second. Often, congealing at ambient temperature results in sufficiently rapid solidification of the droplets to avoid excessive azithromycin ester formation. However, the congealing step often occurs in an enclosed space to simplify collection of the multiparticulates. In such cases, the temperature of the congealing medium (either gas or liquid) will increase over time as the droplets are introduced into the enclosed space, leading to the possible formation of azithromycin esters. Thus, a cooling gas or liquid is often circulated through the enclosed space to maintain a constant congealing temperature. When the carrier used is highly reactive with azithromycin and the time the azithromycin is exposed to the molten carrier must be limited, the cooling gas or liquid can be cooled to below ambient temperature to promote rapid congealing, thus keeping the formation of azithromycin esters to acceptable levels.

Suitable thermal-based processes are disclosed in detail in commonly assigned U.S. patent application Ser. No. ______ (“Improved Azithromycin Multiparticulate Dosage Forms by Melt-Congeal Processes,” Attorney Docket No. PC25015) and Ser. No. ______ (“Extrusion Process for Forming Chemically Stable Multiparticulates,” Attorney Docket No. PC25122) filed concurrently herewith.

While the azithromycin in the multiparticulates can be amorphous or crystalline, it is preferred that at least 70 wt % of the azithromycin in the multiparticulates is crystalline. The crystalline form is preferred because it tends to result in multiparticulates with improved chemical and physical stability.

The inventors have found that when the azithromycin is in a form that contains a volatile species, such as water or a solvent, one key to maintaining the crystalline form during formation of multiparticulates is to maintain a high activity of the volatile species in the carrier, atmosphere or gas with which the composition comes in contact. The activity of the volatile species should be equivalent to or greater than that in the crystalline state. This will ensure that the volatile species present in the crystal form of azithromycin remains at equilibrium with the atmosphere, thus preventing a loss of hydrated water or solvated solvent. For example, if the process for forming the multiparticulates requires that crystalline azithromycin, the crystalline dihydrate, for instance, be exposed to high temperatures (e.g., during a melt- or spray-congeal process), the atmosphere near the azithromycin should be maintained at high humidity to limit the loss of the hydrated water from the azithromycin crystals, and thus a change in the crystalline form of the azithromycin.

The humidity level required is that which is equivalent to or greater than the activity of water in the crystalline state. This can be determined experimentally, for example, using a dynamic vapor sorption apparatus. In this test, a sample of the crystalline azithromycin is placed in a chamber and equilibrated at a constant temperature and relative humidity. The weight of the sample is then recorded. The weight of the sample is then monitored as the relative humidity of the atmosphere in the chamber is decreased. When the relative humidity in the chamber decreases to below the level equivalent to the activity of water in the crystalline state, the sample will begin to lose weight as waters of hydration are lost. Thus, to maintain the crystalline state of the azithromycin, the humidity level should be maintained at or above the relative humidity at which the azithromycin begins to lose weight. A similar test can be used to determine the appropriate amount of solvent vapor required to maintain a crystalline solvate form of azithromycin.

When crystalline azithromycin, such as the dihydrate form, is added to a molten carrier, a small amount of water, on the order of 30 to 100 wt % of the solubility of water in the molten carrier at the process temperature can be added to the carrier to ensure there is sufficient water to prevent loss of the azithromycin dihydrate crystalline form.

Processes to maintain the crystalline form of azithromycin while forming multiparticulates are disclosed more fully in commonly assigned U.S. patent application Ser. No. ______ (“Method for Making Pharmaceutical Multiparticulates,” Attorney Docket No. PC25021), filed concurrently herewith.

The multiparticulates of the present invention may also be post-treated to improve the drug crystallinity and/or the stability of the multiparticulate. In one embodiment, the multiparticulates comprise azithromycin and a carrier having a melting point of Tₘ in ° C.; the multiparticulates are treated after formation by at least one of (i) heating the multiparticulates to a temperature of at least about 35° C. but less than about (Tₘ° C.−10° C.), and (ii) exposing the multiparticulates to a mobility-enhancing agent. Such a post-treatment step results in an increase in drug crystallinity in the multiparticulates, and typically an improvement in at least one of the chemical stability, physical stability, and dissolution stability of the multiparticulates. Post-treatment processes are disclosed more fully in commonly assigned U.S. patent application Ser. No. ______, (“Multiparticulate Compositions with Improved Stability,” Attorney Docket No. PC11900) filed concurrently herewith.

Preferably the azithromycin dosage form comprises multiparticulates, which comprise from about 45 to about 55 wt % azithromycin, from about 43 to about 50 wt % glyceryl behenate, from about 2 to about 5 wt % poloxamer, and an alkalizing agent comprising from about 300 to about 400 mg tribasic sodium phosphate (TSP), and are post-treated by maintaining them at a temperature of about 40° C. at a relative humidity of about 75%, or sealed with water in a container maintained at about 40° C. for 2 days or more. It is more preferred that this dosage form further comprises from about 200 to about 300 mg magnesium hydroxide.

More preferably the azithromycin dosage form comprises multiparticulates, which comprise about 50 wt % azithromycin dihydrate, from about 46 to about 48 wt % Compritol® 888 ATO, from about 2 to about 4 wt % Lutrol® F127 NF, and an alkalizing agent comprising from about 300 to about 400 mg TSP, and are post-treated by maintaining them at a temperature of about 40° C. at a relative humidity of about 75%, or sealed with water in a container maintained at 40° C. for about 5 days to about 3 weeks. It is more preferred that this dosage form further comprises from about 200 to about 300 mg magnesium hydroxide.

Most preferably the azithromycin dosage form comprises multiparticulates, which comprise about 50 wt % azithromycin dihydrate, about 47 wt % Compritol® 888 ATO and about 3 wt % Lutrol® F127 NF, and are post-treated by maintaining them at a temperature of about 40° C. at a relative humidity of about 75%, or sealed with water in a container maintained at 40° C. for about 10 days or more.

### Carriers and Optional Excipients

The multiparticulates comprise a pharmaceutically acceptable carrier and optional excipients. By “pharmaceutically acceptable” is meant the carrier and optional excipients must be compatible with the other ingredients of the composition, and not deleterious to the recipient thereof. The carrier functions as a matrix for the multiparticulate or to affect the rate of release of azithromycin from the multiparticulate, or both. Azithromycin can potentially react with carriers and optional excipients having acidic or ester groups to form esters of azithromycin. Carriers and optional excipients may be characterized as having “low reactivity,” “medium reactivity,” and “high reactivity” in relation to their tendency to form azithromycin esters. In general, ester formation is kept at low levels by selecting low reactivity carriers and optional excipients, and/or by processes that limit the exposure of the azithromycin to medium and high reactive carriers and optional excipients at elevated temperature. Preferably, the concentration of azithromycin esters in the multiparticulates is less than about 1 wt %, based on the total amount of azithromycin present in the multiparticulate. Preferably, the concentration of azithromycin esters is less than about 0.5 wt %, more preferably less than about 0.2 wt %, and most preferably less than about 0.1 wt %. Processes for reducing ester formation are described in more detail in commonly assigned U.S. patent application Ser. No. ______ (“Improved Azithromycin Multiparticulate Dosage Forms by Melt-Congeal Processes,” Attorney Docket No. PC25015), Ser. No. ______ (“Controlled Release Multiparticulates Formed with Dissolution Enhancers,” Attorney Docket No. PC25016), and Ser. No. ______ (“Improved Azithromycin Multiparticulate Dosage Forms by Liquid-Based Processes, Attorney Docket No. PC25018), filed concurrently herewith.

Examples of low reactivity carriers and optional excipients include long-chain alcohols, such as stearyl alcohol, cetyl alcohol, and polyethylene glycol; poloxamers (block copolymers of ethylene oxide and propylene oxide, such as poloxamer 188, poloxamer 237, poloxamer 338, and poloxamer 407); ethers, such as polyoxyethylene alkyl ethers; ether-substituted cellulosics, such as microcrystalline cellulose, hydroxypropyl cellulose, hydroxypropyl methyl cellulose, and ethylcellulose; sugars such as glucose, sucrose, xylitol, sorbitol, and maltitol; and salts such as sodium chloride, potassium chloride, lithium chloride, calcium chloride, magnesium chloride, sodium sulfate, potassium sulfate, sodium carbonate, magnesium sulfate, and potassium phosphate.

Moderate reactivity carriers and optional excipients often contain acid or ester substituents, but relatively few as compared to the molecular weight of the carrier or optional excipient. Examples include long-chain fatty acid esters, such as glyceryl monooleate, glyceryl monostearate, glyceryl palmitostearate, polyethoxylated castor oil derivatives, hydrogenated vegetable oils, glyceryl dibehenate, and mixtures of mono-, di-, and tri-alkyl glycerides; glycolized fatty acid esters, such as polyethylene glycol stearate and polyethylene glycol distearate; polysorbates; and waxes, such as carnauba wax and white and yellow beeswax.

Highly reactive carriers and optional excipients usually have several acid or ester substituents or low molecular weights. Examples include carboxylic acids such as stearic acid, benzoic acid, citric acid, fumaric acid, lactic acid, and maleic acid; short to medium chain fatty-acid esters, such as isopropyl palmitate, isopropyl myristate, triethyl citrate, lecithin, triacetin, and dibutyl sebacate; ester-substituted cellulosics, such as cellulose acetate, cellulose acetate phthalate, hydroxypropyl methyl cellulose phthalate, cellulose acetate trimellitate, and hydroxypropyl methyl cellulose acetate succinate; and acid or ester functionalized polymethacrylates and polyacrylates. Generally, the acid/ester concentration on highly reactive carriers and optional excipients is so high that if these carriers and optional excipients come into direct contact with azithromycin in the formulation, unacceptably high concentrations of azithromycin esters form during processing or storage of the composition. Thus, such highly reactive carriers and optional excipients are preferably only used in combination with a carrier or optional excipient with lower reactivity so that the total amount of acid and ester groups on the carrier and optional excipients used in the multiparticulate is low.

The inventors have found that for multiparticulates with an acceptable amount of azithromycin esters (i.e., less than about 1 wt %), there is a trade-off relationship between the concentration of acid and ester substituents on the carrier and optional excipients and the crystallinity of azithromycin in the multiparticulate. Generally speaking, the greater the crystallinity of azithromycin in the multiparticulate, the greater the degree of the carrier's or optional excipient's acid/ester substitution may be to obtain a multiparticulate with acceptable amounts of azithromycin esters. This relationship may be quantified by the following mathematical expression:
[A]≦0.04/(1−x) (I)
where [A] is the total concentration of acid/ester substitution on the carrier and optional excipients in meq/g azithromycin and is less than or equal to 2 meq/g, and x is the weight fraction of the azithromycin in the composition that is crystalline. When the carrier and optional excipients comprises more than one excipient, the value of [A] refers to the total concentration of acid/ester substitution on all the excipients that make up the carrier and optional excipients, in units of meq/g azithromycin.

For more preferable multiparticulates having less than about 0.5 wt % azithromycin esters, the azithromycin, carrier, and optional excipients will satisfy the following expression:
[A]≦0.02/(1−x). (II)

For more preferable multiparticulates having less than about 0.2 wt % azithromycin esters, the azithromycin, carrier, and optional excipients will satisfy the following expression:
[A]≦0.008/(1−x). (III)

For most preferable multiparticulates having less than about 0.1 wt % azithromycin esters, the azithromycin, carrier, and optional excipients will satisfy the following expression:
[A]≦0.004/(1−x). (IV)

From the foregoing mathematical expressions (I)—(IV) the trade-off between the carrier's and optional excipient's degree of acid/ester substitution and the crystallinity of azithromycin in the composition can be determined.

Carriers used in the melt-congeal process of the present invention will generally make up about 10 wt % to about 95 wt % of the multiparticulate, preferably about 20 wt % to about 90 wt, and more preferably about 40 wt % to about 70 wt %, based on the total mass of the multiparticulate. The carrier is preferably solid at temperatures of about 40° C. The inventors have found that if the carrier is not a solid at 40° C., there can be changes in the physical characteristics of the composition over time, especially when stored at elevated temperatures, such as at 40° C. Thus, it is preferred that the carrier be a solid at a temperature of about 50° C., more preferably at about 60° C. It is also preferred that the carrier have a melting point that is less then the melting point of azithromycin. For example, azithromycin dihydrate has a melting point of 113° C. to 115° C. Thus, when azithromycin dihydrate is used in the multiparticulates of the present invention, it is preferred that the carrier have a melting point that is less than about 113° C.

Examples of carriers suitable for use in the multiparticulates of the present invention include waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate; long-chain alcohols, such as stearyl alcohol, cetyl alcohol, and polyethylene glycol; and mixtures thereof.

The multiparticulates may optionally include a dissolution enhancer. Dissolution enhancers increase the rate of dissolution of the drug from the carrier. In general, dissolution enhancers are amphiphilic compounds and are generally more hydrophilic than the carrier. Dissolution enhancers will generally make up about 0.1 to about 30 wt % of the total mass of the multiparticulate. Exemplary dissolution enhancers include alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; sugars such as glucose, sucrose, xylitol, sorbitol, and maltitol; salts such as sodium chloride, potassium chloride, lithium chloride, calcium chloride, magnesium chloride, sodium sulfate, potassium sulfate, sodium carbonate, magnesium sulfate, and potassium phosphate; amino acids such as alanine and glycine; and mixtures thereof. Preferably, the dissolution enhancer is at least one surfactant, and most preferably, the dissolution enhancer is at least one poloxamer.

While not wishing to be bound by any particular theory or mechanism, it is believed that a dissolution enhancer present in the multiparticulates affects the rate at which the aqueous use environment penetrates the multiparticulate, thus affecting the rate at which azithromycin is released. In addition, such excipients may enhance the azithromycin release rate by aiding in the aqueous dissolution of the carrier itself, often by solubilizing the carrier in micelles. Further details of dissolution enhancers and selection of appropriate excipients for azithromycin multiparticulates are disclosed in commonly assigned U.S. patent application Ser. No. ______ (“Controlled Release Multiparticulates Formed with Dissolution Enhancers,” Attorney Docket No. PC25016), filed concurrently herewith.

Agents that inhibit or delay the release of azithromycin from the multiparticulates can also be included in the carrier. Such dissolution-inhibiting agents are generally hydrophobic. Examples of dissolution-inhibiting agents include: hydrocarbon waxes, such as microcrystalline and paraffin wax; and polyethylene glycols having molecular weights greater than about 20,000 daltons.

Another useful class of excipients that may optionally be included in the multiparticulates include materials that are used to adjust the viscosity of the molten feed used to form the multiparticulates, for example, by a melt-congeal process. Such viscosity-adjusting excipients will generally make up 0 to 25 wt % of the multiparticulate, based on the total mass of the multiparticulate. The viscosity of the molten feed is a key variable in obtaining multiparticulates with a narrow particle size distribution. For example, when a spinning-disc atomizer is employed, it is preferred that the viscosity of the molten mixture be at least about 1 cp and less than about 10,000 cp, more preferably at least 50 cp and less than about 1000 cp. If the molten mixture has a viscosity outside these preferred ranges, a viscosity-adjusting carrier can be added to obtain a molten mixture within the preferred viscosity range. Examples of viscosity-reducing excipients include stearyl alcohol, cetyl alcohol, low molecular weight polyethylene glycol (e.g., less than about 1000 daltons), isopropyl alcohol, and water. Examples of viscosity-increasing excipients include microcrystalline wax, paraffin wax, synthetic wax, high molecular weight polyethylene glycols (e.g., greater than about 5000 daltons), ethyl cellulose, hydroxypropyl cellulose, hydroxypropyl methyl cellulose, methyl cellulose, silicon dioxide, microcrystalline cellulose, magnesium silicate, sugars, and salts.

Other excipients may be added to adjust the release characteristics of the multiparticulates or to improve processing and will typically make up 0 to 50 wt % of the multiparticulate, based on the total mass of the multiparticulate. For example, since the solubility of azithromycin in aqueous solution decreases with increasing pH, a base may be included in the composition to decrease the rate at which azithromycin is released in an aqueous use environment. Examples of bases that can be included in the composition include di- and tribasic sodium phosphate, di- and tribasic calcium phosphate, mono-, di-, and triethanolamine, sodium bicarbonate and sodium citrate dihydrate as well as other oxide, hydroxide, phosphate, carbonate, bicarbonate and citrate salts, including hydrated and anhydrous forms known in the art. Still other excipients may be added to reduce the static charge on the multiparticulates. Examples of such anti-static agents include talc and silicon dioxide. Flavorants, colorants, and other excipients may also be added in their usual amounts for their usual purposes.

In one embodiment, the carrier and one or more optional excipients form a solid solution, meaning that the carrier and one or more optional excipients form a single thermodynamically stable phase. In such cases, excipients that are not solid at a temperature of less than about 40° C. can be used, provided the carrier/excipient mixture is solid at a temperature of up to about 40° C. This will depend on the melting point of the excipients used and the relative amount of carrier included in the composition. Generally, the greater the melting point of one excipient, the greater the amount of a low-melting-point excipient that can be added to the composition while still maintaining a carrier in a solid phase at 40° C.

In another embodiment, the carrier and one or more optional excipients do not form a solid solution, meaning that the carrier and one or more optional excipients form two or more thermodynamically stable phases. In such cases, the carrier/excipient mixture may be entirely molten at processing temperatures used to form multiparticulates or one material may be solid while the other(s) are molten, resulting in a suspension of one material in the molten mixture.

When the carrier and one or more optional excipients do not form a solid solution but one is desired, for example, to obtain a specific controlled-release profile, an additional excipient may be included in the composition to produce a solid solution comprising the carrier, the one or more optional excipients, and the additional excipient. For example, it may be desirable to use a carrier comprising microcrystalline wax and a poloxamer to obtain a multiparticulate with the desired release profile. In such cases a solid solution is not formed, in part due to the hydrophobic nature of the microcrystalline wax and the hydrophilic nature of the poloxamer. By including a small amount of a third excipient, such as stearyl alcohol, in the formulation, a solid solution can be obtained, resulting in a multiparticulate with the desired release profile.

In one aspect, the multiparticulates are in the form of a non-disintegrating matrix. By “non-disintegrating matrix” is meant that at least a portion of the carrier does not dissolve or disintegrate after introduction of the multiparticulates to an aqueous use environment. In such cases, the azithromycin and optionally a portion of one or more of the carriers or optional excipients, for example, a dissolution-enhancer, are removed from the multiparticulate by dissolution. At least a portion of the carrier does not dissolve or disintegrate and is excreted when the use environment is in vivo, or remains suspended in a test solution when the use environment is in vitro. In this aspect, it is preferred that at least a portion of the carrier have a low solubility in the aqueous use environment. Preferably, the solubility of at least a portion of the carrier in the aqueous use environment is less than about 1 mg/mL, more preferably less than about 0.1 mg/mL, and most preferably less than about 0.01 mg/ml. Examples of suitable low-solubility carriers include waxes, such as synthetic wax, microcrystalline wax, paraffin wax, carnauba wax, and beeswax; glycerides, such as glyceryl monooleate, glyceryl monostearate, glyceryl palmitostearate, glyceryl mono-, di- or tribehenates, glyceryl tristearate, glyceryl tripalmitate; and mixtures thereof.

In one embodiment, the multiparticulate comprises (i) about 20 to about 75 wt % azithromycin, (ii) about 25 to about 80 wt % of a carrier, and (iii) about 0.1 to about 30 wt % of a dissolution enhancer based on the total mass of the multiparticulate.

In a more preferred embodiment, the multiparticulate comprises about (i) 35 wt % to about 55 wt % azithromycin; (ii) about 40 wt % to about 65 wt % of an excipient selected from waxes, such as synthetic wax, microcrystalline wax, paraffin wax, carnauba wax, and beeswax; glycerides, such as glyceryl monooleate, glyceryl monostearate, glyceryl palmitostearate, polyethoxylated castor oil derivatives, hydrogenated vegetable oils, glyceryl mono-, di- or tribehenates, glyceryl tristearate, glyceryl tripalmitate and mixtures thereof; and (iii) about 0.1 wt % to about 15 wt % of a dissolution enhancer selected from surfactants, such as poloxamers, polyoxyethylene alkyl ethers, polyethylene glycol, polysorbates, polyoxyethylene alkyl esters, sodium lauryl sulfate, and sorbitan monoesters; alcohols, such as stearyl alcohol, cetyl alcohol and polyethylene glycol; sugars, such as glucose, sucrose, xylitol, sorbitol and maltitol; salts, such as sodium chloride, potassium chloride, lithium chloride, calcium chloride, magnesium chloride, sodium sulfate, potassium sulfate, sodium carbonate, magnesium sulfate and potassium phosphate; amino acids, such as alanine and glycine; and mixtures thereof.

In another embodiment, the multiparticulates comprise (i) azithromycin; (ii) a glyceride carrier having at least one alkylate substituent of 16 or more carbon atoms; and (iii) a poloxamer. At least 70 wt % of the drug in the multiparticulate is crystalline. The choice of these particular carrier excipients allows for precise control of the release rate of the azithromycin over a wide range of release rates. Small changes in the relative amounts of the glyceride carrier and the poloxamer result in large changes in the release rate of the drug. This allows the release rate of the drug from the multiparticulate to be precisely controlled by selecting the proper ratio of drug, glyceride carrier and poloxamer. These matrix materials have the further advantage of releasing nearly all of the drug from the multiparticulate. Such multiparticulates are disclosed more fully in commonly assigned U.S. patent application Ser. No. ______ (“Multiparticulate Crystalline Drug Compositions Having Controlled Release Profiles,” Attorney Docket No. PC25020), filed concurrently herewith.

### EXAMPLES

### Formulation M1

Formulation M1 multiparticulates were made comprising 50 wt % azithromycin dihydrate, 47 wt % COMPRITOL 888 ATO (a mixture of glyceryl mono-, di- and tri-behenates from Gattefossé Corporation of Paramus, N.J.), and 3 wt % LUTROL F127 (pharmaceutical grade poloxamer 407 with an average molecular weight of 9800 to 14,600 daltons from BASF Corporation of Mt. Olive, N.J.) using the following process. First, 5000 g azithromycin dihydrate, 4700 g of the COMPRITOL 888 ATO and 300 g of the LUTROL Fl 27 were blended in a twinshell blender for 20 minutes. This blend was then de-lumped using a Fitzpatrick L1A mill at 3000 rpm, knives forward using a 0.065-inch screen. The mixture was blended again in a twinshell blender for 20 minutes, forming a preblend feed. The preblend feed was delivered to a B&P 19-mm twin-screw extruder (MP19-TC with a 25 UD ratio purchased from B & P Process Equipment and Systems, LLC, Saginaw, Mich.) at a rate of 140 g/min. Water was added to the extruder at a rate of 4 g/min (3 wt % of the preblend feed). The extruder produced a molten mixture consisting of a suspension of the azithromycin dihydrate in the COMPRITOL 888 ATO/LUTROL F127 at a temperature of about 90° C. The feed suspension was then delivered to the center of a spinning-disk atomizer. The spinning disk atomizer, which was custom made, consists of a bowl-shaped stainless steel disk of 10.1 cm (4 inches) in diameter. The surface of the disk is heated with a thin film heater beneath the disk to about 90° C. That disk is mounted on a motor that drives the disk of up to approximately 10,000 RPM. The entire assembly is enclosed in a plastic bag of approximately 8 feet in diameter to allow congealing and to capture microparticulates formed by the atomizer. Air is introduced from a port underneath the disk to provide cooling of the multiparticulates upon congealing and to inflate the bag to its extended size and shape. The surface of the spinning disk atomizer was maintained at 90° C. and the disk was rotated at 5500 rpm while forming the azithromycin multiparticulates.

A suitable commercial equivalent to this spinning disk atomizer is the FX1 100-mm rotary atomizer manufactured by Niro A/S (Soeborg, Denmark).

The mean residence time of azithromycin in the twin-screw extruder was about 60 seconds, and the total time the azithromycin was exposed to the molten suspension was less than about 3 minutes. The particles formed by the spinning-disk atomizer were congealed in ambient air and collected. The so-formed multiparticulates had a diameter of about 180 μm.

The multiparticulates of Formulation M1 were annealed by placing samples of the same in a shallow tray at a depth of about 2 cm, placing the tray in a sealed container, then heating the sealed container in a 40° C. oven for 2 days.

### Formulations M2-M5

For Formulations M2-M5, multiparticulates were made comprising azithromycin dihydrate, COMPRITOL 888 ATO, and PLURONIC F127 (poloxamer 407 from BASF Corporation of Mt. Olive, N.J.), as described for Formulation M1, with the exceptions noted in Table 1. For Formulations M2-M4 the annealing post-treatment step was conducted at 75% relative humidity.

TABLE 1

| | | | H₂O | | | | |
|---|---|---|---|---|---|---|---|
| | MP Formulation | Feed | Feed | Disk | Disk | Batch | Annealing |
| | (wt % Az/ | Rate | Rate | Speed | Temp | Size | (° C./% RH; |
| MP. No. | Comp/Plur) | (g/min) | (wt %) | (rpm) | (° C.) | (g) | days) |
| M1 | 50/47/3 | 140 | 3 | 5500 | 90 | 10,000 | 40/sealed; 2 |
| M2 | 50/46.75/3.25 | 140 | 0 | 5500 | 90 | 738.1 | 40/75; 5 |
| M3 | 50/46.5/3.5 | 140 | 0 | 5500 | 90 | 737.8 | 40/75; 5 |
| M4 | 50/46.25/3.75 | 140 | 0 | 5500 | 90 | 738.6 | 40/75; 5 |
| M5 | 50/46/4 | 140 | 3 | 5500 | 90 | 5000 | 40/sealed; 2 |

Key:
MP = multiparticulate
Az = azithromycin dihydrate
Comp = COMPRITOL 888 ATO
Plur = PLURONIC F127
RH = relative humidity

### Dosing Excipients D1

A blend of excipients was prepared for use in preparing a dosage form with the multiparticulates. Dosing Excipients D1 consisted of NF grades of the following: about 20 g sucrose, about 35 mg hydroxypropyl cellulose, about 35 mg xanthan gum, and about 100 mg colloidal SiO₂; about 210 mg USP grade titanium dioxide; about 180 mg of the alkalizing agent anhydrous tribasic sodium phosphate (TSP); and about 75 mg and about 120 mg of artificial cherry and banana flavoring, respectively.

### Examples 1-5

This example illustrates that the rate of release of azithromycin from a dosage form can be adjusted by varying the amount of poloxamer in the multiparticulate formulation. For Example 1, a multiparticulate dosage form was formed by blending 2000 mgA of multiparticulate Formulation M1 with the Dosing Excipients D1. For Examples 2-5, multiparticulate dosage forms were formed by blending 2000 mgA each of multiparticulate Formulations M2 to M5 with Dosing Excipients D1.

The in vitro rate of release of azithromycin from the multiparticulate dosage forms of Examples 1-5 was then determined using the following procedure. The multiparticulate dosage forms, each containing about 2000 mgA of azithromycin, were placed into individual 125 mL bottles. Next, 60 mL of purified water was added, and the bottle was shaken for 30 seconds. The contents were added to a USP Type 2 dissoette flask equipped with Teflon-coated paddles rotating at 50 rpm. The flask contained 840 mL of a simulated gastric fluid medium comprising 100 mM Na₂HPO₄ buffer, pH 6.0, maintained at 37.0±0.5° C. The bottle was rinsed twice with 20 mL of the buffer from the flask, and the rinse was returned to the flask to make up a 900 mL final volume. A 3 mL sample of the fluid in the flask was then collected at 15, 30, 60, 120, and 180 minutes following addition of the multiparticulates to the flask. The samples were filtered using a 0.45-μm syringe filter prior to analyzing via HPLC (Hewlett Packard 1100, Waters Symmetry C₈ column, 45:30:25 acetonitrile:methanol:25 mM KH₂PO₄ buffer at 1.0 mL/min, absorbance measured at 210 nm with a diode array spectrophotometer). The results of these dissolution tests are given in Table 2.

TABLE 2

| | | | | Azithromycin | Azithromycin |
|---|---|---|---|---|---|
| Ex. | MP | Dosing | Time | Released | Released |
| No. | Formulation | Excipients | (hr) | (mgA) | (%) |
| 1 | M1 | D1 | 0 | 0.0 | 0 |
| | | | 0.25 | 780 | 39 |
| | | | 0.5 | 1280 | 64 |
| | | | 1 | 1720 | 86 |
| | | | 2 | 1880 | 94 |
| | | | 3 | 1900 | 95 |
| 2 | M2 | D1 | 0 | 0.0 | 0 |
| | | | 0.25 | 660 | 33 |
| | | | 0.5 | 1040 | 52 |
| | | | 1 | 1510 | 75 |
| | | | 2 | 1890 | 94 |
| | | | 3 | 1950 | 98 |
| 3 | M3 | D1 | 0 | 0 | 0 |
| | | | 0.25 | 760 | 38 |
| | | | 0.5 | 1170 | 59 |
| | | | 1 | 1650 | 83 |
| | | | 2 | 1940 | 97 |
| | | | 3 | 1980 | 99 |
| 4 | M4 | D1 | 0 | 0 | 0 |
| | | | 0.25 | 850 | 42 |
| | | | 0.5 | 1270 | 64 |
| | | | 1 | 1740 | 87 |
| | | | 2 | 1950 | 97 |
| | | | 3 | 1970 | 99 |
| 5 | M5 | D1 | 0 | 0 | 0 |
| | | | 0.25 | 1030 | 52 |
| | | | 0.5 | 1540 | 77 |
| | | | 1 | 1870 | 93 |
| | | | 2 | 1940 | 97 |
| | | | 3 | 1940 | 97 |

These data show that the amount of the poloxamer dissolution enhancer in the multiparticulates has an effect on the release rate of azithromycin into the dissolution medium.

### Formulation M6

For Formulation M6, multiparticulates were made comprising 50 wt % azithromycin dihydrate, 46 wt % COMPRITOL 888 ATO, and 4 wt % LUTROL F127 as described for Formulation M1, with the exceptions noted in Table 3.

TABLE 3

| | | H₂O | | | | |
|---|---|---|---|---|---|---|
| M6 Formulation | Feed | Feed | Disk | Disk | | Annealing |
| (wt % Az/Comp/ | Rate | Rate | speed | Temp | Batch | (° C./% |
| Lut*) | (g/min) | (wt %) | (rpm) | (° C.) | size (g) | RH; days) |
| 50/46/4 | 120 | 0 | 5500 | 90 | 5868 | 40/75; 5 |

*Lut = LUTROL F127

### Dosing Excipients D2-D7

Blends of alkalizing agents were prepared for use in preparing multiparticulate dosage forms of the multiparticulates. Dosing Excipients D2-D7 each contained 38.7 g sucrose and various amounts of TSP as follows: D2 contained 50 mg TSP; D3 100 mg; D4 264 mg; D5 356 mg; D6 500 mg; and D7 0 mg (as a control).

### Examples 6-10

These examples illustrate that the release rate of azithromycin can be delayed by varying the amount of alkalizing agent in the dosage form. For Examples 6-10, multiparticulate dosage forms were prepared by blending 2000 mgA of multiparticulate Formulation M6 with Dosing Excipients D2-D6, as indicated in Table 4. Control C1 was prepared in the same manner as in Examples 6-10 with the exception that Dosing Excipient D7 was used. The in vitro rates of release of azithromycin from the multiparticulate dosage forms of Examples 6-10 were measured as in Examples 1-5, except that the dissolution flask contained a final volume of 750 mL of 0.01 N HCl held at 37.0±0.5° C. The results of these dissolution tests are given in Table 4.

TABLE 4

| | | | | Azithromycin | Azithromycin |
|---|---|---|---|---|---|
| Ex. | MP | Dosing | Time | Released | Released |
| No. | Formulation | Excipients | (hr) | (mg) | (%) |
| 6 | M6 | D2 | 0 | 0 | 0 |
| | | | 0.08 | 350 | 17 |
| | | | 0.25 | 760 | 38 |
| | | | 0.5 | 1130 | 57 |
| | | | 1 | 1440 | 72 |
| | | | 2 | 1610 | 81 |
| | | | 3 | 1680 | 84 |
| 7 | M6 | D3 | 0 | 0 | 0 |
| | | | 0.08 | 340 | 17 |
| | | | 0.25 | 740 | 37 |
| | | | 0.5 | 1020 | 51 |
| | | | 1 | 1260 | 63 |
| | | | 2 | 1420 | 71 |
| | | | 3 | 1520 | 76 |
| 8 | M6 | D4 | 0 | 0 | 0 |
| | | | 0.08 | 300 | 15 |
| | | | 0.25 | 630 | 31 |
| | | | 0.5 | 880 | 44 |
| | | | 1 | 1160 | 58 |
| | | | 2 | 1400 | 70 |
| | | | 3 | 1480 | 74 |
| 9 | M6 | D5 | 0 | 0 | 0 |
| | | | 0.08 | 250 | 12 |
| | | | 0.25 | 490 | 24 |
| | | | 0.5 | 710 | 35 |
| | | | 1 | 920 | 46 |
| | | | 2 | 1120 | 56 |
| | | | 3 | 1240 | 62 |
| 10 | M6 | D6 | 0 | 0 | 0 |
| | | | 0.08 | 160 | 8 |
| | | | 0.25 | 340 | 17 |
| | | | 0.5 | 480 | 24 |
| | | | 1 | 640 | 32 |
| | | | 2 | 850 | 42 |
| | | | 3 | 1010 | 50 |
| C1 | M6 | D7 | 0 | 0 | 0 |
| | | | 0.08 | 420 | 21 |
| | | | 0.25 | 860 | 43 |
| | | | 0.5 | 1160 | 58 |
| | | | 1 | 1460 | 73 |
| | | | 2 | 1660 | 83 |
| | | | 3 | 1720 | 86 |

The data in Table 4 confirm that multiparticulate Formulation M6 when dosed in a dosing vehicle containing an alkalizing agent to alter the pH of the acidic dissolution media provides controlled release of azithromycin within the desired range of release rates. Specifically, the multiparticulate dosage forms of Examples 6-10, which included the alkalizing agent TSP in the dosing excipients, all released less than 40 wt % of the azithromycin at 0.25 hour following administration to the acidic test medium. On the other hand, Control C1, made using multiparticulate Formulation M6 and Dosing Excipient D7 containing no alkalizing agent, released 43 wt % of the azithromycin at 0.25 hour following administration to the gastric test medium.

### Dosing Excipients D8-D11

Blends of excipients were prepared for use in preparing dosage forms of the multiparticulates. Dosing Excipients D8 and D9 each contained 38.7 g sucrose and one other excipient as follows: D8 contained 100 mg of the weak base sodium carbonate; D9 contained 50 mg magnesium hydroxide. Dosing Excipient D10 contained 1.0 g of MAALOX® (liquid, regular strength, smooth cherry, available from Novartis Consumer Health, Inc., Parsippany, N.J.). The density of MAALOX® is 1.0775 g/mL. Thus, according to the product information, 1.0 g of MAALOX® contains 37.1 mg aluminum hydroxide, 37.1 mg of magnesium hydroxide, and 3.7 mg simethicone, in addition to inactive ingredients. Dosing Excipient D11 contained 80 mL ENSURE PLUS® (a liquid nutritional supplement from Ross Products Division of Abbott Laboratories, Inc., Abbott Park, Ill.).

### Examples 11-14

These examples illustrate that the release rate of azithromycin can be adjusted by varying the excipients included in the dosage form. Multiparticulate dosage forms were prepared by blending 2000 mgA of multiparticulate Formulation M6 with Dosing Excipients D8-D11, as indicated in Table 5. The in vitro azithromycin release rates were measured as in Examples 1-5, except that the flask contained a final volume of 750 mL of 0.01 N HCl held at 37.0±0.5° C. The results of these dissolution tests are given in Table 5.

TABLE 5

| | | | | Azithromycin | Azithromycin |
|---|---|---|---|---|---|
| Ex. | MP | Dosing | Time | Released | Released |
| No. | Formulation | Excipients | (hr) | (mg) | (%) |
| 11 | M6 | D8 | 0 | 0 | 0 |
| | | | 0.08 | 130 | 10 |
| | | | 0.25 | 270 | 20 |
| | | | 0.5 | 430 | 32 |
| | | | 1 | 590 | 45 |
| | | | 2 | 1170 | 59 |
| | | | 3 | 1360 | 68 |
| 12 | M6 | D9 | 0 | 0 | 0 |
| | | | 0.08 | 210 | 16 |
| | | | 0.25 | 470 | 35 |
| | | | 0.5 | 670 | 50 |
| | | | 1 | 830 | 62 |
| | | | 2 | 1460 | 73 |
| | | | 3 | 1580 | 79 |
| 13 | M6 | D10 | 0 | 0 | 0 |
| | | | 0.08 | 220 | 17 |
| | | | 0.25 | 490 | 36 |
| | | | 0.5 | 650 | 49 |
| | | | 1 | 830 | 62 |
| | | | 2 | 1440 | 72 |
| | | | 3 | 1520 | 76 |
| 14 | M6 | D11 | 0 | 0 | 0 |
| | | | 0.08 | 90 | 7 |
| | | | 0.25 | 170 | 13 |
| | | | 0.5 | 250 | 19 |
| | | | 1 | 400 | 30 |
| | | | 2 | 870 | 44 |
| | | | 3 | 1030 | 52 |

### Control Dosage Form C2

Control Dosage Form C2 consisted of two ZITHROMAX® (Pfizer, Inc., Groton, Conn.) immediate release sachets for oral suspension, which is commercially available in a single dose packet containing azithromycin dihydrate equivalent to 1000 mgA azithromycin, 88 mg TSP, and the inactive ingredients previously noted.

### Control Dosage Form C3

Control Dosage Form C3 consisted of eight ZITHROMAX® tablets, each containing azithromycin dihydrate equivalent to 250 mgA azithromycin and the inactive ingredients previously noted.

Rates of release of azithromycin from Control Dosage Forms C2 and C3 were measured in vitro as in Examples 1-5, except that the flask contained a final volume of 750 mL of 0.01 N HCl held at 37.0±0.5° C. For Control C2, 2 packets were tested to obtain a total of 2000 mgA azithromycin and 176 mg TSP. For Control C3, 8 tablets were tested to obtain a total of 2000 mgA azithromycin. Control C3 tests were performed with and without the addition of 176 mg TSP to the test medium. The results of these dissolution tests are given in Table 6.

The rate of release of azithromycin from Control Dosage Form C2 was measured in vitro as in Examples 1-5 using a dissolution medium of 100 mM Na₂HPO₄ at pH 6.0. Two packets were tested to obtain a total of 2000 mgA azithromycin. The results of these dissolution tests are also given in Table 6.

TABLE 6

| | | | | | Azithro- |
|---|---|---|---|---|---|
| | Control | | | Azithromycin | mycin |
| Control | Dosage | Dissolution | Time | Released | Released |
| No. | Form | Medium | (hr) | (mg) | (%) |
| C2 | 2 packets | 0.01 N HCl | 0 | 0 | 0 |
| | (contains | | 0.08 | 1050 | 79 |
| | 2000 mgA | | 0.25 | 1180 | 88 |
| | azithromycin | | 0.5 | 1230 | 92 |
| | and 176 | | 1 | 1270 | 95 |
| | mg TSP) | | 2 | 1950 | 97 |
| | | | 3 | 1960 | 98 |
| C2 | 2 packets | 100 mM | 0 | | 0 |
| | (contains | Na₂HPO₄ | 0.25 | 2000 | 100 |
| | 2000 mgA | | 0.5 | 2000 | 100 |
| | azithromycin | | 1 | 2000 | 100 |
| | and 176 | | 2 | 2000 | 100 |
| | mg TSP) | | 3 | 2000 | 100 |
| C3 | 8 tablets | 0.01 N HCl | 0 | 0 | 0 |
| | | | 0.08 | 1100 | 55 |
| | | | 0.25 | 1480 | 74 |
| | | | 0.5 | 1600 | 80 |
| | | | 1 | 1700 | 85 |
| | | | 2 | 1720 | 86 |
| | | | 3 | 1700 | 85 |
| C3 | 8 tablets | 0.01 N HCl | 0 | 0 | 0 |
| | | with | 0.08 | 1040 | 52 |
| | | 176 mg | 0.25 | 1380 | 69 |
| | | TSP | 0.5 | 1500 | 75 |
| | | | 1 | 1580 | 79 |
| | | | 2 | 1600 | 80 |
| | | | 3 | 1620 | 81 |

### Formulation M7

Multiparticulates were made comprising 50 wt % azithromycin dihydrate, 47 wt % COMPRITOL 888 ATO, and 3 wt % PLURONIC F127 in the same manner as for Formulation M1, with the exceptions noted in Table 7, and a Leistritz 27 mm twin-screw extruder (Model ZSE 27, American Leistritz Extruder Corporation, Somerville, N.J.) was used to form the molten mixture.

TABLE 7

| | | H₂O | | | |
|---|---|---|---|---|---|
| M7 Formulation | Feed | Feed | Disk | Disk | Annealing |
| (wt % Az/ | Rate | Rate | Speed | Temp | (° C./% |
| Comp/Plur) | (g/min) | (wt %) | (rpm) | (° C.) | RH; days) |
| 50/47/3 | 140 | 0 | 5500 | 90 | 40/75; 5 |

### Dosing Excipients D12-D13

Two blends of excipients D12-D13 were prepared from the same grades of Dosing Excipient D1 and having the following compositions: D12 contained 19.36 g sucrose, 352 mg TSP, 250 mg magnesium hydroxide, 67 mg hydroxypropyl cellulose, 67 mg xanthan gum, 200 mg colloidal silicon dioxide, 400 mg titanium dioxide, 140 mg cherry flavoring and 230 mg banana flavoring; D13 contained the same excipients in the same amounts, but without the magnesium hydroxide.

### Examples 15-16

Multiparticulate dosage forms for Examples 15-16 were prepared by mixing 2000 mgA of multiparticulate Formulation M7 with Dosing Excipients D12 and D13, respectively.

### Example 17

Clinical studies were conducted to evaluate the pharmacokinetics of the multiparticulate dosage forms of Examples 15-16, each of which contained at least one alkalizing agent, as compared to an azithromycin immediate release dosage form.

The in vivo pharmacokinetics of the dosage forms of Examples 15-16 were evaluated in 32 fasting, healthy human subjects in a randomized, open-label, parallel group, two way cross-over study. On Day 1, eight subjects received the Example 15 azithromycin multiparticulate dosage form and eight subjects received the Example 16 azithromycin multiparticulate dosage form. As controls, two groups (A and B) of eight subjects each received an immediate release control (C2) consisting of two single dose packets of azithromycin dihydrate for oral suspension (ZITHROMAX®, Pfizer Inc., New York, N.Y.) wherein each dose contained 1048 mg azithromycin dehydrate (equivalent to 1000 mgA azithromycin), 88 mg TSP and the inactive ingredients previously noted.

Specifically, 2000 mgA doses of either the azithromycin formulations of Examples 15-16 or of the ZITHROMAX® were dosed based upon a computer-generated randomization for each of the two treatment groups.

To dose the Examples 15 and 16 formulations, 60 mL of water was added to the bottle containing the dosage form and was shaken for 30 seconds. The entire contents of the bottle were administered directly into the subject's mouth. An additional 60 mL of water was added to rinse the bottle and the rinse was administered to the subject's mouth. An additional 120 mL of water was administered using a dosing cup.

To dose the control dosage form C2, the contents of one ZITHROMAX® single dose packet were emptied into a cup containing 60 mL of water. The mixture was stirred and was administered to the subject's mouth. An additional 60 mL of water was used to rinse the cup and the rinse was administered. This procedure was repeated for a second ZITHROMAX® single dose packet.

All subjects were orally dosed after an overnight fast and were then required to refrain from lying down, eating or drinking beverages other than water during the first 4 hours after dosing.

Blood samples (5 mL each) were withdrawn from each subject prior to dosing, and at 0.5, 1, 2, 3, 4, 6, 8, 12, 16, 24, 36, 48, 72 and 96 hours after dosing. Serum azithromycin concentrations were determined using the HPLC assay described in Shepard el al., 565 J. Chromatography 321 (1991). Total systemic exposure to azithromycin was determined by measuring the area under the curve (AUC) for each subject in the group and then by calculating a mean AUC over the 96 hours for the group. Cₘₐₓ (the highest serum azithromycin concentration achieved in a subject) and Tₘₐₓ (the time at which Cₘₐₓ was achieved) were both determined.

On Day 15, the procedure was repeated. The two 8-subject groups, who received the control dosage forms on Day 1, were then dosed with the azithromycin multiparticulate dosage forms of Examples 15 or 16. Likewise, the two 8-subject groups who previously received the azithromycin multiparticulate dosage forms on Day 1, were then dosed with the control dosage form C2.

The resulting serum pharmacokinetic data are reported in Table 8.

TABLE 8

| | | | Cₘₐₓ | | Tₘₐₓ | | AUC | |
|---|---|---|---|---|---|---|---|---|
| | | | (μg/mL) | | (hr) | | (μg · hr/mL) | |
| Ex. | | Dosing | Geometric | % | Arithmetic | | Geometric | |
| No. | Formulation | Excipients | Mean | CV | Mean | SD | Mean | % CV |
| 15 | M7 | D12 | 0.82 | 26 | 4.13 | 1.6 | 15.75 | 40 |
| 16 | M7 | D13 | 0.92 | 36 | 2.94 | 1.7 | 13.81 | 35 |
| C2 | 2 packets | None | 1.90 | 49 | 1.56 | 0.7 | 19.03 | 24 |
| (Group A) | ZITHROMAX ® | | | | | | | |
| C2 | 2 packets | None | 2.09 | 36 | 1.13 | 0.3 | 18.98 | 22 |
| (Group B) | ZITHROMAX ® | | | | | | | |

CV = Coefficient of Variation
SD = Standard Deviation

Based upon the results in Table 8, the Example 15 azithromycin multiparticulate dosage form provided a bioavailability that was 83% relative to the immediate release control dosage form while the Example 16 azithromycin multiparticulate dosage form provided a bioavailability that was 73% relative to the immediate release control dosage form. The data also showed that the ratios of the maximum serum concentration of azithromycin provided by the multiparticulate dosage forms of Examples 15 and 16 to the maximum serum concentration of azithromycin provided by the immediate release control dosage forms were 0.43 and 0.44, respectively. In addition, the time to achieve the maximum concentration was longer for the azithromycin multiparticulate dosage forms than for the immediate release control dosage forms.

### Example 18

Clinical studies were conducted to evaluate the in vivo gastrointestinal (GI) toleration of the multiparticulate dosage forms of Examples 15-16, each of which contained at least one alkalizing agent, as compared to an azithromycin immediate release control dosage form.

The study consisted of a randomized, parallel group study. Specifically, 106 healthy human subjects were orally administered the Example 15 formulation, 106 healthy human subjects were orally administered the Example 16, and 108 healthy human subjects were each administered immediate release controls (C2) of two single dose 1000 mgA ZITHROMAX® packets, using the procedures described in Example 17.

GI adverse events, such as diarrhea, nausea, and vomiting, were monitored for 24 hours following administration of each dosage form by asking subjects non-leading questions at the following approximate hours after dosing: 1, 2, 4, 6, 8, 12, and 24.

The incidence of GI adverse events experienced by the subjects are reported in Table 9.

TABLE 9

| | | | Subjects with GI Adverse Events | | |
|---|---|---|---|---|---|
| | | | (expressed as % of total number | | |
| | | | of subjects tested) [relative degree | | |
| Ex. | MP | Dosing | of improvement in toleration] | | |
| No. | Formulation | Excipients | Diarrhea | Nausea | Vomiting |
| 15 | M7 | 12 | 17.9 (1.6) | 17.0 (3.2) | 2.8 (9.3) |
| 16 | M7 | 13 | 23.6 (1.2) | 17.0 (3.2) | 3.8 (6.8) |
| C2 | 2 packets | None | 27.8 | 54.6 | 25.9 |
| | ZITHROMAX ® | | | | |

The results in Table 9 show that the multiparticulate dosage forms of the invention provided substantially improved toleration relative to the immediate release control dosage form C2. Specifically, the multiparticulate dosage form of Examples 15-16 provided a relative degree of improvement in toleration with respect to vomiting of 9.3 (25.9%÷2.8%) and 6.8 (25.9%÷3.8%), respectively, relative to the control C2 and in toleration with respect to nausea of 3.2 (54.6%÷17.0%) relative to the control C2.

### Formulation M8

Multiparticulates were made comprising 50 wt % azithromycin dihydrate, 48 wt % COMPRITOL 888 ATO, and 2 wt % poloxamer 407 (PLURONIC F127) in the same manner as for Formulation M7, with the exceptions noted in Table 10.

TABLE 10

| | | H₂O | | | |
|---|---|---|---|---|---|
| M8 Formulation | Feed | Feed | Disk | Disk | |
| (wt % Az/ | Rate | Rate | Speed | Temp | (° C./% |
| Comp/Plur) | (g/min) | (wt %) | (rpm) | (° C.) | RH; days) |
| 50/48/2 | 140 | 0 | 5500 | 90 | 40/75; 5 |

### Dosing Excipients D14

Dosing Excipients D14 were prepared having the same excipients in the same amounts as for Dosing Excipient D12, with the exception that there was only 110 mg of colloidal silicon dioxide.

### Example 19

This Example illustrates that dosage forms of the present invention can be obtained by using a small amount of poloxamer in the multiparticulate formulation. Multiparticulate dosage forms were prepared by mixing 2000 mgA of multiparticulate Formulation M8 with Dosing Excipients D14. The in vitro azithromycin release rates were measured in two separate tests (1) using 0.01 N HCl as the dissolution medium as in Example 6, and (2) using 100 mM Na₂HPO₄ as the dissolution medium as in Example 1. The results of these dissolution tests are given in Table 11.

TABLE 11

| | | | | Azithro- | Azithro- |
|---|---|---|---|---|---|
| | | | | mycin | mycin |
| MP | Dosing | Dissolution | Time | Released | Released |
| Formulation | Excipients | Medium | (hr) | (mg) | (%) |
| M8 | D14 | 0.01 N HCl | 0 | 0 | 0 |
| | | | 0.25 | 280 | 14 |
| | | | 0.5 | 620 | 31 |
| | | | 1 | 1180 | 59 |
| | | | 2 | 1820 | 91 |
| | | | 3 | 2000 | 100 |
| M8 | D14 | 100 mM | 0 | 0 | 0 |
| | | Na₂HPO₄ | 0.25 | 460 | 23 |
| | | | 0.5 | 760 | 38 |
| | | | 1 | 1180 | 59 |
| | | | 2 | 1640 | 82 |
| | | | 3 | 1840 | 92 |

The data in Table 11 confirm that the multiparticulate dosage form of Example 19 is within the scope of the invention.

### Example 20

Clinical studies were conducted to evaluate the pharmacokinetics of the multiparticulate dosage form of Example 19, as compared to an azithromycin immediate release dosage form. The immediate release control was control dosage form C2 of Examples 11-14. The dosage forms were evaluated using the procedures outlined in Example 17. The results of these tests are shown in Table 12.

TABLE 12

| | | | Cₘₐₓ | | Tₘₐₓ | | AUC | |
|---|---|---|---|---|---|---|---|---|
| | | | (μg/mL) | | (hr) | | (μg · hr/mL) | |
| Example | | Dosing | Geometric | % | Arithmetic | | Geometric | % |
| No. | Formulation | Excipients | Mean | CV | Mean | SD | Mean | CV |
| 19 | M8 | D14 | 0.86 | 26 | 4.88 | 1.86 | 13.6 | 25 |
| C2 | 2 packets | None | 2.10 | 42 | 1.25 | 0.58 | 15.3 | 24 |
| | ZITHROMAX ® | | | | | | | |

The results show that the multiparticulate dosage form of Example 19 provided a relative bioavailability that was 89% relative to the immediate release control dosage form C2. The data also show that the ratio of the maximum serum concentration of azithromycin provided by the multiparticulate dosage form of Example 19 to the maximum serum concentration of azithromycin provided by the immediate release control dosage form C2 was 0.41. In addition, the time to achieve the maximum concentration was longer for the multiparticulate dosage forms than for the immediate release control dosage form C2.

In addition, the incidence of GI adverse events experienced by 16 of the subjects tested was evaluated using the procedures outlined in Example 18. The results of this study showed that none of the subjects experienced vomiting and only 3 subjects (19%) experienced nausea when administered the dosage form of Example 19, whereas 1 subject (6%) experienced vomiting and 8 subjects (50%) experienced nausea. Thus, the sustained release azithromycin multiparticulate dosage form of Example 19 also provided improved toleration relative to the immediate release control dosage form C2.

### Formulation M9

Multiparticulates were made comprising 50 wt % azithromycin dihydrate, 47 wt % COMPRITOL 888 ATO, and 3 wt % LUTROL F127 using the following procedure. First, 15 kg azithromycin dihydrate, 14.1 kg of the COMPRITOL 888 ATO and 0.9 kg of the LUTROL Fl 27 were weighed and passed through a Quadro 194S Comil mill in that order. The mill speed was set at 600 rpm. The mill was equipped with a No. 2C-075-H050/60 screen (special round), a No. 2C-1607-049 flat-blade impeller, and a 0.225-inch spacer between the impeller and screen. The mixture was blended using a Servo-Lift 100-L stainless-steel bin blender rotating at 20 rpm, for a total of 500 rotations, forming a preblend feed.

The preblend feed was delivered to a Leistritz 50 mm twin-screw extruder (Model ZSE 50, American Leistritz Extruder Corporation, Somerville, N.J.) at a rate of 25 kg/hr. The extruder was operated in co-rotating mode at about 300 rpm, and interfaced with a melt/spray-congeal unit. The extruder had nine segmented barrel zones and an overall extruder length of 36 screw diameters (1.8 m). Water was injected into barrel number 4 at a rate of 8.3 g/min (2 wt %). The extruders rate of extrusion was adjusted so as to produce a molten feed suspension of the azithromycin dihydrate in the COMPRITOL 888 ATO/LUTROL F127 at a temperature of about 90° C.

The feed suspension was delivered to a spinning-disk atomizer rotating at 7600 rpm, the surface of which was maintained at 90° C. The maximum total time the azithromycin was exposed to the molten suspension was less than about 10 minutes. The particles formed by the spinning-disk atomizer were cooled and congealed in the presence of cooling air circulated through the product collection chamber. The mean particle size was determined to be 188 μm using a Horiba LA-910 particle size analyzer. Samples of the multiparticulates were also evaluated by PXRD, which showed that about 99% of the azithromycin in the multiparticulates was in the crystalline dihydrate form.

The so-formed multiparticulates were post-treated by placing samples in sealed barrels that were then placed in a controlled atmosphere chamber at 40° C. for 3 weeks.

### Example 21

Multiparticulate dosage forms were prepared by mixing 2000 mgA of multiparticulate Formulation M9 with Dosing Excipients D14. The in vitro azithromycin release rates were measured in two separate tests (1) using 0.01 N HCl as the dissolution medium as in Example 6, and (2) using 100 mM Na₂HPO₄ as the dissolution medium as in Example 1. The results of these dissolution tests are given in Table 13.

TABLE 13

| | | | | Azithro- | Azithro- |
|---|---|---|---|---|---|
| | | | | mycin | mycin |
| MP | Dosing | Dissolution | Time | Released | Released |
| Formulation | Excipients | Medium | (hr) | (mg) | (%) |
| M9 | D14 | 0.01 N HCl | 0 | 0 | 0 |
| | | | 0.25 | 520 | 26 |
| | | | 0.5 | 700 | 35 |
| | | | 1 | 920 | 46 |
| M9 | D14 | 100 mM | 0 | 0 | 0 |
| | | Na₂HPO₄ | 0.25 | 320 | 16 |
| | | | 0.5 | 600 | 30 |
| | | | 1 | 1040 | 52 |
| | | | 2 | 1560 | 78 |
| | | | 3 | 1780 | 89 |

The data in Table 13 confirm that the multiparticulate dosage form of Example 21 is within the scope of the invention.

### Formulation M10

Multiparticulates are formed by the following process. A mixture comprising 20 wt % azithromycin dihydrate and 80 wt % COMPRITOL 888 ATO is formed by blending azithromycin dihydrate and the COMPRITOL 888 ATO in a twin-shell blender for 10 minutes. This blend is then de-lumped in a Fitzpatrick L1A mill at 3000 rpm with knives forward using a 0.060″ screen. The blend is then mixed for an additional 10 minutes in a twin-shell blender.

Particles are then made by granulating this mixture in a fluidized bed granulator. The mixture is charged to the granulator and air is circulated to fluidize the mixture. Water, adjusted to a pH greater than 7 by addition of trisodium phosphate, is sprayed into the fluidized bed to form particles. The particles are then dried in a tray dryer to remove a substantial portion of the water from the particles, without dehydrating the azithromycin dihydrate. The resulting particles are sieved to isolate particles with mean diameters ranging from 100 μm to 500 μm.

### Example 22

Multiparticulate dosage forms are prepared by mixing 2000 mgA of multiparticulate Formulation M10 with Dosing Excipients D14. The in vitro azithromycin release rates are measured using 0.01 N HCl as the dissolution medium as in Example 6, and using 100 mM Na₂HPO₄ as the dissolution medium as in Example 1 to demonstrate that the dosage form of Example 22 is within the scope of the invention.

### Formulation M11

A mixture is formed as in Formulation M10 and particles are then made by wet-granulating the mixture using a mortar and pestle. Water, adjusted to a pH greater than 7 by the addition of trisodium phosphate, is used as the granulation fluid. The particles are then dried in a tray dryer to remove a substantial portion of the water from the particles, without dehydrating the azithromycin dihydrate, and sieved to isolate particles with mean diameters ranging from 100 μm to 500 μm.

### Example 23

Multiparticulate dosage forms are prepared by mixing 2000 mgA of multiparticulate Formulation M11 with Dosing Excipients D14. The in vitro azithromycin release rates are measured using 0.01 N HCl as the dissolution medium as in Example 6, and using 100 mM Na₂HPO₄ as the dissolution medium as in Example 1 to demonstrate that the dosage form of Example 23 is within the scope of the invention.

### Formulations M12-M14

Multiparticulate Formulations M12-M14 were made comprising 50 wt % azithromycin dihydrate, 47 wt % COMPRITOL 888 ATO, and 3 wt % LUTROL F127 as outlined for Formulation M1 with the exceptions noted in Table 14. The disk speed was varied to produce multiparticulates with various mean diameters.

TABLE 14

| | MP | | H₂O | | | | Mean |
|---|---|---|---|---|---|---|---|
| | Formulation | Feed | Feed | Disk | Disk | Annealing | MP |
| | (wt % Az/ | Rate | Rate | Speed | Temp | (° C./% RH; | Diameter |
| MP. No. | Comp/Lut) | (g/min) | (wt %) | (rpm) | (° C.) | days) | (μm) |
| M12 | 50/47/3 | 131 | 2 | 5500 | 90 | 40° C./75% | 188 |
| | | | | | | RH sealed; | |
| | | | | | | 21 days | |
| M13 | 50/47/3 | 131 | 2 | 4800 | 90 | 40° C./75% | 204 |
| | | | | | | RH sealed; | |
| | | | | | | 21 days | |
| M14 | 50/47/3 | 131 | 2 | 4100 | 90 | 40° C./75% | 227 |
| | | | | | | RH sealed; | |
| | | | | | | 21 days | |

### Examples 24-26

These examples illustrate that the rate of release of azithromycin from multiparticulate dosage forms can be adjusted by varying the size of the multiparticulates. Multiparticulate dosage forms were prepared by mixing 2000 mgA of multiparticulate Formulation M12 with Dosing Excipients D14 (Example 24), multiparticulate Formulation M13 with Dosing Excipients D14 (Example 25), and multiparticulate Formulation M14 with Dosing Excipients D14 (Example 26).

The in vitro azithromycin release rates were measured using 100 mM Na₂HPO₄ as the dissolution medium as in Example 1. The results of these dissolution tests are given in Table 15, and show that dosage forms of Examples 24-26 are within the scope of the invention. The data also show that as the mean diameter of the multiparticulates was increased, the rate of release of azithromycin from the multiparticulates was decreased.

TABLE 15

| | | | | Azithromycin | Azithromycin |
|---|---|---|---|---|---|
| Ex. | MP | Dosing | Time | Released | Released |
| No. | Formulation | Excipients | (hr) | (mgA) | (%) |
| 24 | M12 | D14 | 0 | 0 | 0 |
| | | | 0.25 | 560 | 28 |
| | | | 0.5 | 920 | 46 |
| | | | 1 | 1400 | 70 |
| | | | 2 | 1800 | 90 |
| | | | 3 | 1900 | 95 |
| 25 | M13 | D14 | 0 | 0 | 0 |
| | | | 0.25 | 520 | 26 |
| | | | 0.5 | 860 | 43 |
| | | | 1 | 1320 | 66 |
| | | | 2 | 1740 | 87 |
| | | | 3 | 1860 | 93 |
| 26 | M14 | D14 | 0 | 0 | 0 |
| | | | 0.25 | 500 | 25 |
| | | | 0.5 | 800 | 40 |
| | | | 1 | 1240 | 62 |
| | | | 2 | 1680 | 84 |
| | | | 3 | 1840 | 92 |

### Formulation M15

Multiparticulates were made comprising 50 wt % azithromycin dihydrate, 47 wt % COMPRITOL 888 ATO, and 3 wt % LUTROL F127 using the following procedure. First, 140 kg azithromycin dihydrate was weighed and passed through a Quadro Comil 196S with a mill speed of 900 rpm. The mill was equipped with a No. 2C-075-H050/60 screen (special round, 0.075″), a No. 2F-1607-254 impeller, and a 0.225 inch spacer between the impeller and screen. Next, 8.4 kg of the LUTROL Fl 27 and then 131.6 kg of the COMPRITOL 888 ATO were weighed and passed through a Quadro 194S Comil mill. The mill speed was set at 650 rpm. The mill was equipped with a No. 2C-075-R03751 screen (0.075”), a No. 2C-1601-001 impeller, and a 0.225-inch spacer between the impeller and screen. The mixture was blended using a Gallay 38 cubic foot stainless-steel bin blender rotating at 10 rpm for 40 minutes, for a total of 400 rotations, forming a preblend feed.

The preblend feed was delivered to a Leistritz 50 mm twin-screw extruder (Model ZSE 50, American Leistritz Extruder Corporation, Somerville, N.J.) at a rate of about 20 kg/hr. The extruder was operated in co-rotating mode at about 100 rpm, and interfaced with a melt/spray-congeal unit. The extruder had five segmented barrel zones and an overall extruder length of 20 screw diameters (1.0 m). Water was injected into barrel number 2 at a rate of 6.7 g/min (2 wt %). The extruder's rate of extrusion was adjusted so as to produce a molten feed suspension of the azithromycin dihydrate in the COMPRITOL 888 ATO/LUTROL F127 at a temperature of about 90° C.

The feed suspension was delivered to the spinning-disk atomizer described in connection with Formulation M1, rotating at 6400 rpm and maintained at a temperature of about 90° C. The maximum total time the azithromycin was exposed to the molten suspension was less than 10 minutes. The particles formed by the spinning-disk atomizer were cooled and congealed in the presence of cooling air circulated through the product collection chamber. The mean particle size was determined to be about 200 μm using a Malvern particle size analyzer.

The so-formed multiparticulates were post-treated by placing a sample in a sealed barrel that was then placed in a controlled atmosphere chamber at 40° C. for 10 days. Samples of the post-treated multiparticulates were evaluated by PXRD, which showed that about 99% of the azithromycin in the multiparticulates was in the crystalline dihydrate form.

### Example 27

Multiparticulate dosage forms were prepared by mixing 2000 mgA of multiparticulate Formulation M15 with Dosing Excipients D14. The in vitro azithromycin release rates were measured using 100 mM Na₂HPO₄ as the dissolution medium as in Example 1. The results of these dissolution tests are given in Table 16.

TABLE 16

| | | | Azithromycin | Azithromycin |
|---|---|---|---|---|
| MP | Dosing | Time | Released | Released |
| Formulation | Excipients | (hr) | (mg) | (%) |
| M15 | D14 | 0 | 0 | 0 |
| | | 0.25 | 720 | 37 |
| | | 0.5 | 1200 | 60 |
| | | 1 | 1700 | 85 |
| | | 2 | 1920 | 96 |
| | | 3 | 1980 | 99 |

The data in Table 16 confirm that the multiparticulate dosage form of Example 27 is within the scope of the invention.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A process for the formation of multiparticulates comprising the steps:
(a) forming a molten mixture comprising azithromycin, a pharmaceutically acceptable carrier and an optional dissolution enhancer;
(b) delivering said molten mixture of step (a) to an atomizing means to form droplets from said mixture;
(c) congealing said droplets from step (b) to form said multiparticulates; and
(d) post-treating said multiparticulates so as to increase the degree of crystallinity of said azithromycin in said multiparticulates.

2. The process of claim 1, wherein said molten mixture of step (a) is formed in an extruder.

3. The process of claim 1 wherein said molten mixture is formed at a processing temperature that is at least about 10° C. above the melting point of said carrier.

4. The process of claim 1 wherein said molten mixture is a suspension of said azithromycin in said carrier and wherein at least 70 wt % of said azithromycin is crystalline.

5. The process of claim 1 wherein said atomizing means is selected from the group consisting of spinning-disk atomizers, pressure nozzles, two-fluid nozzles, ultrasonic nozzles and mechanical vibrating nozzles.

6. The process of claim 5 wherein said atomizing means is a spinning-disk atomizer.

7. The process of claim 1 wherein said multiparticulates have a mean diameter of from 100 to 300 microns.

8. The process of claim 1 wherein said azithromycin is in a crystal form that comprises a volatile species and wherein said volatile species is added during at least one of steps (a), (b), and (c).

9. The process of claim 1 wherein step (d) comprises heating said multiparticulates to a temperature of at least about 35° C. and less than about (Tₘ−10° C.), where Tₘ is the melting point of said carrier.

10. The process of claim 1 wherein step (d) comprises exposing said multiparticulates to a mobility-enhancing agent.

11. The process of claim 1 wherein said multiparticulates further comprise a mobility-enhancing agent.

12. The process of claim 11 wherein step (d) comprises the steps:
(i) placing said multiparticulates in a sealed container; and
(ii) heating said sealed container at a temperature of not more than about (Tₘ-10° C.), where Tₘ is the melting point of said carrier.

13. The process of claim 12 wherein said mobility-enhancing agent is water.

14. The process of claim 1 wherein water is added during step (a) to said molten mixture.

15. The process of claim 1 wherein said azithromycin is in the dihydrate form.

16. The process of claim 1 wherein following step (d) said azithromycin is at least 90 wt % crystalline.

17. The process of claim 1 wherein said azithromycin is present in an amount of from about 20 to about 75 wt % of said multiparticulate.

18. The process of claim 1 wherein said carrier is present in an amount of from about 25 to about 80 wt % of said multiparticulate.

19. The process of claim 1 wherein said dissolution enhancer is present in an amount of from about 0.1 to about 30 wt % of said multiparticulate.

20. The process of claim 1 wherein said azithromycin is present in an amount of from about 20 to about 75 wt % of said multiparticulate, said carrier is present in an amount of from about 25 to about 80 wt % of said multiparticulate, and said dissolution enhancer is present in an amount of from about 0.1 to about 30 wt % of said multiparticulate.

21. The process of claim 1 wherein said azithromycin is present in an amount of from about 35 to about 55 wt % of said multiparticulate, said carrier comprises a glyceride and is present in an amount of from about 40 to about 65 wt % of said multiparticulate, and said dissolution enhancer comprises a poloxamer present in an amount of from about 0.1 to about 15 wt % of said multiparticulate.

22. The process of claim 1 wherein said carrier is selected from the group consisting of waxes, glycerides, long-chain alcohols, and mixtures thereof.

23. The process of claim 22 wherein said carrier is selected from the group consisting of synthetic wax, microcrystalline wax, paraffin wax, carnauba wax, beeswax, glyceryl monooleate, glyceryl monostearate, glyceryl palmitostearate, polyethoxylated castor oil derivatives, hydrogenated vegetable oils, glyceryl mono-, di-, and tribehenates, glyceryl tristearate, glyceryl tripalmitate, stearyl alcohol, cetyl alcohol, polyethylene glycol, and mixtures thereof.

24. The process of claim 23 wherein said carrier is a mixture of glyceryl mono-, di-, and tribehenates.

25. The process of claim 1 wherein said dissolution enhancer is selected from the group consisting of alcohols, surfactants, sugars, salts, amino acids, and mixtures thereof.

26. The process of claim 25 wherein said dissolution enhancer is selected from the group consisting of stearyl alcohol, cetyl alcohol, polyethylene glycol, poloxamers, docusate salts, polyoxyethylene alkyl ethers, polyoxyethylene castor oil derivatives, polysorbates, polyoxyethylene alkyl esters, sodium lauryl sulfate, sorbitan monoesters, glucose, sucrose, xylitol, sorbitol, maltitol, sodium chloride, potassium chloride, lithium chloride, calcium chloride, magnesium chloride, sodium sulfate, potassium sulfate, sodium carbonate, magnesium sulfate, potassium phosphate, alanine, glycine, and mixtures thereof.

27. The process of claim 26 wherein said dissolution enhancer is a surfactant.

28. The process of claim 27 wherein said dissolution enhancer is a poloxamer.

29. The process of claim 1 wherein said carrier is a mixture of glyceryl mono-, di-, and tribehenates, and said dissolution enhancer is a poloxamer.

30. The product of the process of claim 1.

31. The product of the process of claim 29.

32. A process for the formation of multiparticulates comprising the steps:
(a) forming in an extruder a molten mixture comprising azithromycin dihydrate, a mixture of glyceryl mono-, di-, and tribehenates, a poloxamer, and water;
(b) delivering said molten mixture of step (a) to a spinning-disk atomizer to form droplets from said mixture;
(c) congealing said droplets from step (b) to form said multiparticulates;
(d) placing said multiparticulates in a sealed container; and
(e) heating said sealed container at a temperature of about 40° C. for about 10 days so as to increase the degree of crystallinity of said azithromycin dihydrate in said multiparticulates.

33. The product of the process of claim 32.
